(19) United States
(12) Patent Application Publication
(10) Pub. No.: US 2008/0199518 A1
(43) Pub. Date: Aug. 21, 2008

(54) Controlled-release formulation of piperazine-piperidine antagonists and agonists of the 5-HT1A receptor having enhanced intestinal dissolution

(75) Inventors: Mannching Sherry Ku, Thiells, NY (US); Wendy Ann Dulin, Tuxedo, NY (US); Yanning Lin, Lake Hiawatha, NJ (US)

Correspondence Address: WilmerHale/Wyeth, 60 STATE STREET, BOSTON, MA, 02109, US

(73) Assignee: Wyeth, Madison, NJ (US)

(21) Appl. No.: 11/986,991

(22) Filed: Nov. 27, 2007

Related U.S. Application Data
(60) Provisional application No. 60/861,409, filed on Nov. 28, 2006.

(51) Int. Cl. A61K 9/52; A61K 9/14; A61K 31/496; A61P 25/00; A61K 9/26

(52) U.S. Cl. 424/459; 424/490; 514/253.06; 424/495; 424/497; 424/468

(57) ABSTRACT

The present invention relates to controlled-release beads comprising diquinoline-substituted piperazine-piperidine compounds, such as 5-fluoro-8-{4-[4-[(6-methoxyquinolin-8-yl)piperazin-1-yl]piperidin-1-yl}quinoline, or pharmaceutically acceptable salts thereof; to multiple particulate formulations comprising such beads; to methods of preparing such beads; and to methods of treating 5-HT_{1A}-related disorders using such beads and/or multiple particulate formulations.

### CROSS REFERENCE TO RELATED APPLICATIONS

This application claims the benefit of priority under 35 U.S.C. §119(e) to U.S. Provisional Patent Application No. 60/861,409 filed on Nov. 28, 2006 and is hereby incorporated by reference in its entirety.

### FIELD OF THE INVENTION

The present invention relates to pharmaceutical compositions of piperazine-piperidine compounds including controlled release pharmaceutical compositions. The compounds are useful as 5-HT_{1A} binding agents, particularly as 5-HT_{1A} receptor antagonists and agonists.

### BACKGROUND

Certain N-aryl-piperazine derivatives possess pharmaceutical activity. In particular, certain N-aryl piperazine derivatives act on the central nervous system (CNS) by binding to 5-HT receptors. In pharmacological testing, it has been shown that the certain N-aryl-piperazine derivatives bind to receptors of the 5-HT_{1A} type. Many of the N-aryl piperazine derivatives exhibit activity as 5-HT_{1A} antagonists. See, for example, W. C. Childers, et al., J. Med. Chem., 48: 3467-3470 (2005), U.S. Pat. Nos. 6,465,482, 6,127,357, 6,469,007, and 6,586,436, and PCT Publication No. WO 97/03982, the disclosures of which are incorporated herein by reference in their entireties.

Pharmaceutical compounds that interact with the 5-HT_{1A} receptor are useful to treat a wide variety of central nervous system disorders, such as cognition disorders, anxiety disorders, and depression. Certain piperazine-piperidine compounds have shown utility as 5-HT_{1A} receptor antagonists, agonists and partial antagonist/agonists. See, for example, US 2007/0027160, entitled “Piperazine-Piperidine Antagonists And Agonists Of The 5-HT_{1A} Receptor,” which is incorporated by reference in its entirety. For example, some diquinoline-substituted piperazine-piperidine compounds, such as 5-fluoro-8-{4-[4-[(6-methoxyquinolin-8-yl)piperazin-1-yl]piperidin-1-yl}quinoline, have been demonstrated to be antagonists of the 5-HT_{1A} receptor. During development of 5-fluoro-8-{4-[4-[(6-methoxyquinolin-8-yl)piperazin-1-yl]piperidin-1-yl}quinoline, it was discovered that the compound has a pH-dependent solubility. Over the pH range of about 2.4 to about 8.9, the solubility ranges from about 2.2 mg/mL to about 36 ng/mL, respectively. It is considered to have very low water solubility (approximately 0.04 μg/mL) and is nearly insoluble around the neutral pH of the lower gastrointestinal tract (less than about 1 μg/mL at pH levels greater than about 6). Although the trisuccinate salt of 5-fluoro-8-{4-[4-[(6-methoxyquinolin-8-yl)piperazin-1-yl]piperidin-1-yl}quinoline was found to have higher aqueous solubility that the free base at approximately 1 mg/mL, it still has a pH-dependent solubility with the solubility being at the sub-microgram per milliliter level at pH greater than about six. Permeability characteristics of the molecule in animal studies indicated that this compound has high permeability. Therefore, the rate systemic input of 5-fluoro-8-{4-[4-[(6-methoxyquinolin-8-yl)piperazin-1-yl]piperidin-1-yl}quinoline will be dependent on the dissolution or solubilization of the compound. In addition, in preclinical studies 5-fluoro-8-{4-[4-[(6-methoxyquinolin-8-yl)piperazin-1-yl]piperidin-1-yl}quinoline has demonstrated a short half-life in Sprague-Dawley rats (t_{1/2} of about one hour at 0.3 mg/kg IV bolus). A controlled-release formulation to reduce the dose frequency of 5-fluoro-8-{4-[4-[(6-methoxyquinolin-8-yl)piperazin-1-yl]piperidin-1-yl}quinoline would be beneficial for improving compliance and convenience. However, this type of formulation presents the challenge of maintaining the dissolution of the compound in the lower gastrointestinal tract where solubility is very low.

### SUMMARY

The present invention relates to controlled-release beads comprising diquinoline-substituted piperazine-piperidine compounds, such as 5-fluoro-8-{4-[4-[(6-methoxyquinolin-8-yl)piperazin-1-yl]piperidin-1-yl}quinoline, or pharmaceutically acceptable salts thereof; to multiple particulate formulations comprising such beads; to methods of preparing such beads; and to methods of treating 5-HT_{1A}-related disorders using such beads and/or multiple particulate formulations. Formulation of 5-fluoro-8-{4-[4-[(6-methoxyquinolin-8-yl)piperazin-1-yl]piperidin-1-yl}quinoline is particularly challenging due to the pH dependent solubility of the compound, as well as the changing pH environment of the gastrointestinal tract. If the compound is slowly released from the formulation as it travels through the gastrointestinal tract (e.g., in a conventional sustained release formulation), it will be dissolved in the upper gastrointestinal tract, as solubility is high for this compound in the low pH environment of the stomach. However, as the formulation enters the lower gastrointestinal tract (i.e., the small intestine), solubility is low given the increased pH of this environment. The inventors have discovered that by applying various release-controlling membranes, as well as coatings containing the pharmacological agent in one or more different formulations, a sustained release formulation is achieved that results in release of the pharmacological agent at particular points in the gastrointestinal tract.

In one aspect, the pharmaceutical compositions described herein include controlled-release bead comprising:

(i) a core unit of a substantially water-soluble or water-swellable inert material;

(ii) a first layer on the core unit comprising a pharmacological agent, an acidifier and optionally a binder;

(iii) a second layer of sustained-release coat covering the first layer;

(iv) a third layer of enteric coat on the second layer; and

(v) optionally, an outermost layer comprising the pharmacological agent and optionally a binder.

In another aspect, the pharmaceutical compositions described herein include controlled-release bead comprising:

(i) a core unit comprising a mixture of a substantially water-soluble or water-swellable inert material, a pharmacological agent, an acidifier and optionally a binder;

(ii) a first layer of sustained-release coat on the core unit;

(iii) a second layer of enteric coat covering the first layer; and

(iv) optionally, an outermost layer comprising the pharmacological agent and optionally a binder.

In yet another aspect, the pharmaceutical compositions described herein include a multiple particulate formulation comprising a plurality of beads as described hereinabove. In certain embodiments, the multiple particulate formulation is a capsule or tablet.

In a further aspect, the pharmaceutical compositions described herein include a multiple particulate formulation comprising:

(A) at least one first bead comprising:

(i) a core unit of a substantially water-soluble or water-swellable inert material;

(ii) a first layer on the core unit comprising a pharmacological agent, an acidifier, and an optional binder;

(iii) a second layer of sustained-release coat covering the first layer; and

(iv) a third layer of enteric coat on the second layer; and

(B) at least one second bead comprising the pharmacological agent optionally covered by a sustained-release coat.

In another aspect, the pharmaceutical compositions described herein include a multiple particulate formulation comprising:

(A) at least one first bead comprising:

(i) a core unit comprising a mixture of a substantially water-soluble or water-swellable inert material, a pharmacological agent, an acidifier and an optional binder;

(ii) a first layer of sustained-release coat on the core unit; and

(iii) a second layer of enteric coat covering the first layer; and

(B) at least one second bead comprising the pharmacological agent optionally covered by a sustained-release coat.

In certain embodiments, the pharmacological agent is a compound of Formula I or a pharmaceutically acceptable salt thereof as described herein. In certain other embodiments, the pharmacological agent is 5-fluoro-8-{4-[4-(6-methoxyquinolin-8-yl)piperazin-1-yl]piperidin-1-yl}quinoline trisuccinate.

In a further aspect, the present invention provides a method for preparing a controlled-release bead, the method comprising:

(a) providing a core unit of a substantially water-soluble or water-swellable inert material;

(b) applying a first layer comprising a pharmacological agent, an acidifier and optionally a binder to the core unit;

(c) applying a second layer of sustained-release coat to cover the first layer;

(d) applying a third layer of enteric coat onto the second layer; and

(e) optionally, applying an outermost layer comprising the pharmacological agent and optionally a binder onto the third layer.

In one aspect, the present the present invention provides a controlled-release bead, the method comprising:

(a) providing a core unit by combining a substantially water-soluble or water-swellable inert material with a pharmacological agent, an acidifier and optionally a binder;

(c) applying a first layer of sustained-release coat to cover the core unit;

(d) applying a second layer of enteric coat onto the first layer; and

(e) optionally, applying an outermost layer comprising the pharmacological agent and optionally a binder onto the second layer.

In yet another aspect, the present the present invention provides a method for treating a 5-HT_{1A}-related disorder to a patient in need thereof, the method comprising administering to the patient a therapeutically effective amount of a controlled-release bead or a multiple particulate formulation as described herein.

### BRIEF DESCRIPTION OF THE DRAWINGS

FIG. 1 is a schematic representation of a cross-section of a four-layer coated bead as described herein.

FIG. 2 is a graphical representation depicting the dissolution of a pharmaceutical composition containing four-layer coated beads versus time.

FIG. 3 is a schematic representation of a cross-section of a tri-layer coated bead as described herein.

FIG. 4 is a schematic representation of a cross-section of a bi-layer coated bead as described herein.

FIG. 5 is a graphical representation depicting the dissolution of two pharmaceutical compositions described herein versus time.

FIG. 6 is a graphical representation depicting the solubility of 5-fluoro-8-{4-[4-[(6-methoxyquinolin-8-yl)piperazin-1-yl]piperidin-1-yl}quinoline (mg/mL) as a fuiction of pH.

FIG. 7 is a graphical representation depicting the dissolution of (a) a pharmaceutical composition containing a sustained release polymer and citric acid and (b) a pharmaceutical composition containing a sustained release polymer with no citric acid. The graphical representation depicts the percentage of the compound dissolved as a function of time.

FIG. 8 is a graphical representation depicting the dissolution of (a) a pharmaceutical composition including sustained release coated beads and (b) a pharmaceutical composition including beads coated with a sustained release coat and an enteric coat. The graphical representation depicts the percentage of the compound dissolved as a function of time.

FIG. 9 is a graphical representation depicting the dissolution of (a) a pharmaceutical composition including a mixture of beads coated with a sustained release coat and an enteric coat and (b) a pharmaceutical composition including beads coated with an enteric coat. The graphical representation depicts the percentage of the compound dissolved as a function of time.

### DETAILED DESCRIPTION

The term “(C₁-C₆)-alkyl” as used herein refers to a linear or branched, saturated hydrocarbon having from 1 to 6 carbon atoms. Representative (C₁-C₆)-alkyl groups include, but are not limited to, methyl, ethyl, propyl, isopropyl, butyl, sec-butyl, tert-butyl, pentyl, isopentyl, neopentyl, hexyl, isohexyl, and neohexyl. In one embodiment, the (C₁-C₆)-alkyl group is optionally substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “(C₂-C₆)-alkeny” as used herein refers to a linear or branched hydrocarbon having from 2 to 6 carbon atoms and having at least one carbon-carbon double bond. In one embodiment, the (C₂-C₆)-alkenyl has one double bond. In another embodiment, the (C₂-C₆)-alkenyl has two double bonds. The (C₂-C₆)-alkenyl moiety may exist in the E or Z conformation and the compounds of the present invention include both conformations. In one embodiment, the (C₂-C₆)-alkenyl group is optionally substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “(C₂-C₆)-alkynyl” as used herein refers to a linear or branched hydrocarbon having from 2 to 6 carbon atoms and having at least one carbon-carbon triple bond. In one embodiment, the (C₂-C₆)-alkenyl group is optionally substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

“(C₁-C₆)-haloalkyl” refers to a C₁-C₆ alkyl group, as defined above, wherein one or more of the C₁-C₆ alkyl group's hydrogen atoms has been replaced with —F, —Cl, —Br or —I. Representative examples of an alkylhalo group include, but are not limited to, —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃, —C(CH₃)₂(CH₂Cl), —CH₂CH₂CH₂CH₂CH₂CH₂Br, and —CH₂CH₂CH₂CH₂CH₂CH₂I.

The term “administer”, “administering”, or “administration”, as used herein refers to either directly administering a compound or pharmaceutically acceptable salt of the compound or a composition to an animal, or administering a prodrug derivative or analog of the compound or pharmaceutically acceptable salt of the compound or composition to the animal, which can form an equivalent amount of active compound within the animal's body.

The term “animal” as used herein includes, without limitation, a human, mouse, rat, guinea pig, dog, cat, horse, cow, pig, monkey, chimpanzee, baboon, or rhesus. In one embodiment, the animal is a mammal. In another embodiment, the animal is a human.

The term “conditions effective to” as used herein refers to synthetic reaction conditions that will be apparent to those skilled in the art of synthetic organic chemistry.

The term “effective amount” as used herein refers to an amount of a compound or pharmaceutically acceptable salt of a compound that, when administered to an animal, is effective to prevent, to at least partially ameliorate, or to cure, a condition from which the animal suffers or is suspected to suffer.

The term “halogen” as used herein refers to fluorine, chlorine, bromine, and iodine.

The term “pharmaceutically acceptable salt”, as used herein, refers to salts derived from organic and inorganic acids of a compound of the present invention. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a compound of the present invention.

The term “substantially free of its corresponding opposite enantiomer” as used herein means that the compound contains no more than about 10% by weight of its corresponding opposite enantiomer. In other embodiments, the compound that is substantially free of its corresponding opposite enantiomer contains no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term “5-HT_{1A}-related disorder” as used herein refers to a condition that is mediated through the 5-HT_{1A} receptor. In some embodiments, a 5-HT_{1A}-related disorder is a condition for which it would be beneficial to prevent activation of the 5-HT_{1A} receptor. In other embodiments, a 5-HT_{1A}-related disorder is a condition for which it would be beneficial to activate the 5-HT_{1A} receptor. In one embodiment, a 5-HT_{1A}-related disorder affects the central nervous system (i.e., a CNS-related disorder). Exemplary 5-HT_{1A}-related disorders include, without limitation, depression, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol); behavioral addictions such as an addiction to gambling; and ocular disorders such as glaucoma and ischemic retinopathy; sexual dysfunction associated with drug treatment (e.g., sexual dysfunction associated with SSRI's).

One nonlimiting example of a 5-HT_{1A}-related disorder is a cognition-related disorder (e.g., cognitive dysfunction). Exemplary cognition-related disorders include, without limitation, mild cognitive impairment (MCD, dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the present invention.

Another nonlimiting example of a 5-HT_{1A}-related disorder is an anxiety-related disorder. Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post-traumatic stress disorder, premenstrual dysphoric disorder, social anxiety disorder, eating disorders such as anorexia nervosa and bulimia nervosa, vasomotor flushing, and phobias, including social phobia, agoraphobia, and specific phobias. Substance addition includes, without limitation, drug, alcohol or nicotine addiction.

“Cₘₐₓ,” “Tₘₐₓ,” and “AUC” values reported herein, unless stated as being “mean” values, refer to the values observed in an individual patient. Moreover, Cₘₐₓ, Tₘₐₓ, and AUC values, unless otherwise stated, may be values observed at steady state when dosing at regular time intervals (e.g., every 12 hours) for multiple days (e.g., multiple dose administration) or values for a single dose administration.

In certain embodiments, the present invention provides a pharmaceutically effective amount of a pharmacological agent and an acidifier. In some embodiments, the pharmaceutical compositions further include one or more of a sustained-release coat and/or one or more of an enteric coat.

In some embodiments, the pharmaceutical compositions also include excipients, such as an optional binder, an optional pH adjustment agent, an optional glidant, an optional plasticizer, and an optional surfactant.

The active pharmacological agent is a compound or pharmaceutical salt of a compound of the Formula I:

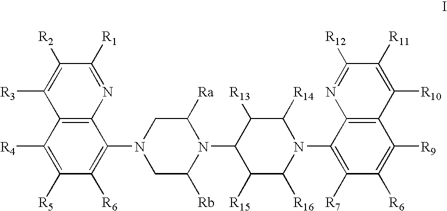

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H, linear or branched (C₁-C₆)-alkyl, (C₁-C₆)-haloalkyl, (C₂-C₆)-alkenyl, or (C₂-C₆)-alkynyl.

In one embodiment, in the compound of Formula I, above:

R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H, linear or branched (C₁-C₆)-alkyl, (C₁-C₆)-haloalkyl, (C₂-C₆)-alkenyl, or (C₂-C₆)-alkynyl.

In one embodiment, R₁ is —H, (C₁-C₆)-alkyl, —OR₂₅, halogen, or —CF₃. In another embodiment, R₁ is —H, (C₁-C₆)-alkyl, —OR₂₅, halogen, or —CF₃ and one of R₁₃, R₁₄, R₁₅, and R₁₆ is —H, (C₁-C₆)-alkyl, —OR₂₅, or halogen. In a further embodiment, R₁ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₇, R₈, R₉, R₁₀, R₁₁, and R₁₂ are each hydrogen. In yet another embodiment, R₁ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₂, R₃, R₄, R₅, R₆, R₇, R₈, R₉, R₁₀, R₁₁, and R₁₂ are each hydrogen. In one embodiment, R₁ is —H, (C₁-C₆)-alkyl, —OR₂₅, halogen or —CF₃ and R₂, R₃, R₄, R₅, R₆, R₇, R₈, R₉, R₁₀, R₁₁, R₁₂, R₁₃, R₁₄, R₁₅, and R₁₆ are each hydrogen.

In one embodiment, R₄ is —H, (C₁-C₆)-alkyl, —OR₂₅, halogen, or —CF₃. In another embodiment, R₄ is —H, (C₁-C₆)-alkyl, —OR₂₅, halogen, or —CF₃ and one of R₁₃, R₁₄, R₁₅, and R₁₆ is —H, (C₁-C₆)-alkyl, —OR₂₅, or halogen. In a further embodiment, R₄ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₇, R₈, R₉, R₁₀, R₁₁, and R₁₂ are each hydrogen. In yet another embodiment, R₄ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halgen, and R₁, R₂, R₃, R₅, R₆, R₇, R₈, R₉, R₁₀, R₁₁, and R₁₂ are each hydrogen. In one embodiment, R₄ is —H, (C₁-C₆)-alkyl, —OR₂₅, halogen or —CF₃ and R₁, R₂, R₃, R₅, R₆, R₇, R₈, R₉, R₁₀, R₁₁, R₁₂, R₁₃, R₁₄, R₁₅, and R₁₆ are each hydrogen.

In one embodiment, R₅ is —H, (C₁-C₆)-alkyl, —OR₂₅, halogen, or —CF₃. In another embodiment, R₅ is —H, (C₁-C₆)-alkyl, —OR₂₅, halogen, or —CF₃ and one of R₁₃, R₁₄, R₁₅, and R₁₆ is —H, (C₁-C₆)-alkyl, —OR₂₅, or halogen. In a further embodiment, R₅ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₇, R₈, R₉, R₁₀, R₁₁, and R₁₂ are each hydrogen. In yet another embodiment, R₅ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen; and R₁, R₂, R₃, R₄, R₆, R₇, R₈, R₉, R₁₀, R₁₁, and R₁₂ are each hydrogen. In one embodiment, R₅ is —H, (C₁-C₆)-alkyl, —OR₂₅, halogen or —CF₃ and R₁, R₂, R₃, R₄, R₆, R₇, R₈, R₉, R₁₀, R₁₁, R₁₂, R₁₃, R₁₄, R₁₅, and R₁₆ are each hydrogen. In a further embodiment, one of R₁₃, R₁₄, R₁₅, and R₁₆, is —H, (C₁-C₆)-alkyl, halogen, —CF₃, or —OR₂₅; R₅ is —H, (C₁-C₆)-alkyl, —OR₂₅, halogen, or —CF₃; and the remaining R groups are each hydrogen.

In one embodiment, R₈ is —H, (C₁-C₆)-alkyl, —OR₂₅, halogen, —CF₃, —NO₂ or —CN. In another embodiment, R₈ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; Rₐ and R_{b} are each independently —H or —CH₃; and each remaining R group is hydrogen. In a further embodiment, R₈ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and each remaining R group is hydrogen. In one embodiment, R₈ is —H, (C₁-C₆)-alkyl, —OR₂₅, halogen, —CF₃, —NO₂ or —CN and one of R₄ or R₅ is —H, (C₁-C₆)-alkyl, —OR₂₅, halogen, or —CF₃, and each remaining R group is hydrogen. In one embodiment R₈ is —H, (C₁-C₆)-alkyl, —OR₂₅, halogen, —CF₃, —NO₂ or —CN; and all other R groups are each hydrogen. In one embodiment, R₈ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and each remaining R group is hydrogen.

In one embodiment, R₉ is —H, (C₁-C₆)-alkyl, —OR₂₅, halogen, —CF₃, —NO₂ or —CN. In another embodiment, R₉ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and each remaining R group is hydrogen. In a further embodiment, R₉ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and each remaining R group is hydrogen. In one embodiment, R₉ is —H, (C₁-C₆)alkyl, —OR₂₅, halogen, —CF₃, —NO₂ or —CN and one of R₄ or R₅ is —H, (C₁-C₆)-alkyl, —OR₂₅, halogen, or —CF₃, and each remaining R group is hydrogen. In one embodiment R₉ is —H, (C₁-C₆)-alkyl, —OR₂₅, halogen, —CF₃, —NO₂ or —CN; and all other R groups are each hydrogen. In one embodiment, R₉ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and each remaining R group is hydrogen.

In one embodiment, R₇ is —H, (C₁-C₆)-alkyl, —OR₂₅, halogen, —CF₃, —NO₂ or —CN. In one embodiment, R₇ is —H, (C₁-C₆)-alkyl or halogen. In one embodiment, R₇ is (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and each remaining R group is hydrogen. In one embodiment, R₇ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and each remaining R group is hydrogen.

In one embodiment, R₁₀ is —H, (C₁-C₆)-alkyl, —OR₂₅, halogen, —CF₃, —NO₂ or —CN. In one embodiment, R₁₀ is —H, (C₁-C₆)-alkyl, —OR₂₅, halogen, or —CF₃. In one embodiment, R₁₀ is —H, —CH₃, —OCH₃, —F or —CF₃. In one embodiment, R₁₀ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and each remaining R group is hydrogen. In one embodiment, R₁₀ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and each remaining R group is hydrogen.

In one embodiment, R₁₁ is —H, (C₁-C₆)-alkyl, —OR₂₅, halogen, —CF₃, —NO₂ or —CN. In one embodiment, R₁₁ is —H, (C₁-C₆)-alkyl, halogen, or —CF₃. In one embodiment, R₁₁ is —CH₃, —F or —CF₃. In one embodiment, R₁₁ is —H, (C₁-C₆)-alkyl, —OR₂₅, halogen, —CF₃, or —NO₂. In one embodiment, R₁₁ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and each remaining R group is hydrogen. In one embodiment, R₁₁ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and each remaining R group is hydrogen.

In one embodiment, R₁₂ is —H, (C₁-C₆)-alkyl, —OR₂₅, halogen, —CF₃, —NO₂ or —CN. In one embodiment, R₁₂ is —H, (C₁-C₆)-alkyl, halogen, or —CF₃. In one embodiment, R₁₂ is —CH₃, —F or —CF₃. In one embodiment, R₁₂ is —H, (C₁-C₆)-alkyl, —OR₂₅, halogen, —CF₃, or —NO₂. In one embodiment, R₁₂ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and each remaining R group is hydrogen. In one embodiment, R₁₂ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and each remaining R group is hydrogen.

In one embodiment, R₅ is —H, (C₁-C₆)-alkyl, —OR₂₅, halogen, or —CF₃ and one of R₇, R₈, R₉, R₁₀, R₁₁ and R₁₂ is —H, (C₁-C₆)-alkyl, —OR₂₅, halogen, —CF₃, —NO₂ or —CN. In another embodiment, R₅ is —H, (C₁-C₆)-alkyl, —OR₂₅, halogen, or —CF₃ and one of R₇, R₈, R₉, R₁₀, R₁₁, R₁₂ is —H, (C₁-C₆)-alkyl, —OR₂₅, halogen, —CF₃, —NO₂ or —CN; and each remaining R group is hydrogen. In some embodiments, R₅ is —H, (C₁-C₆)-alkyl, —OR₂₅, halogen, or —CF₃ and R₉ is —H, (C₁-C₆)-alkyl, —OR₂₅, halogen, —CF₃, —NO₂ or —CN; and each remaining R group is hydrogen. In one embodiment, R₅ is —OR₂₅ and one of R₇, R₈, R₉, R₁₀, R₁₁, and R₁₂ is —H, (C₁-C₆)-alkyl, —OR₂₅, halogen, —CF₃, —NO₂ or —CN. In one embodiment, R₅ is —OR₂₅ and R₉ is halogen. In one embodiment, R₅ is —OR₂₅ and R₉ is halogen and each remaining R group is hydrogen.

In a further embodiment, R₅ is —H, (C₁-C₆)-alkyl, —OR₂₅, halogen, or —CF₃; one of R₇, R₈, R₉, R₁₀, R₁₁, and R₁₂ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and each remaining R group is hydrogen.

In a further embodiment, R₅ is —H, (C₁-C₆)-alkyl, —OR₂₅, halogen, or —CF₃; two of R₇, R₈, R₉, R₁₀, R₁₁, and R₁₂ are each independently —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and each remaining R group is hydrogen.

In a further embodiment, R₅ is —H, (C₁-C₆)-alkyl, —OR₂₅, halogen, or —CF₃; three of R₇, R₈, R₉, R₁₀, R₁₁, and R₁₂ are each independently —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and each remaining R group is hydrogen.

In one embodiment, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; and two of R₁₀, R₁₁ and R₁₂ are each independently —H, (C₁-C₆)-alkyl, —OR₂₅, halogen, —CF₃, —NO₂ or —CN. In another embodiment, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; two of R₁₀, R₁₁, R₁₂ are each independently —H, (C₁-C₆)-alkyl, —OR₂₅, halogen, —CF₃, —NO₂ or —CN; and each remaining R group is hydrogen. In some embodiments, R₅ is —OR₂₅; R₉ is halogen; two of R₁₀, R₁₁, and R₁₂ are each independently —H, (C₁-C₆)-alkyl, —OR₂₅, halogen, —CF₃, —NO₂ or —CN; and each remaining R group is hydrogen. In some embodiments, R₅ is —OCH₃; R₉ is halogen; two of R₁₀, R₁₁, and R₁₂ are each independently —H, (C₁-C₆)-alkyl, —OR₂₅, halogen, —CF₃, —NO₂ or —CN; and each remaining R group is hydrogen.

In some embodiments, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; R₁₀ and R₁₂ are each independently —H, (C₁-C₆)-alkyl, —OR₂₅, halogen, —CF₃, —NO₂ or —CN; and each remaining R group is hydrogen. In some embodiments, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; R₁₀ and R₁₁ are each independently —H, (C₁-C₆)-alkyl, —OR₂₅, halogen, —CF₃, —NO₂ or —CN; and each remaining R group is hydrogen. In some embodiments, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; R₁₁ and R₁₂ are each independently —H, (C₁-C₆)-alkyl, —OR₂₅, halogen, —CF₃, —NO₂ or —CN; and each remaining R group is hydrogen.

In some embodiments, R₅ is —H or —OR₂₅, R₉ is —H or halogen, R₁₀ and R₁₂ are each independently, —H, halogen, or —CF₃; and each remaining R group is hydrogen.

In one embodiment, R₄ is —H, (C₁-C₆)-alkyl, —OR₂₅, halogen, or —CF₃ and one of R₇, R₈, R₉, R₁₀, R₁₁, and R₁₂ is —H, (C₁-C₆)-alkyl, —OR₂₅, halogen, —CF₃, —NO₂ or —CN. In another embodiment, R₄ is —H, (C₁-C₆)-alkyl, —OR₂₅, halogen, or —CF₃; one of R₇, R₈, R₉, R₁₀, R₁₁, and R₁₂ is —H, (C₁-C₆)-alkyl, —OR₂₅, halogen, —CF₃, —NO₂ or —CN; and each remaining R group is hydrogen. In a further embodiment, R₄ is —H, (C₁-C₆)-alkyl, —OR₂₅, halogen, or —CF₃; one of R₇, R₈, R₉, R₁₀, R₁₁; R₁₂ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and each remaining R group is hydrogen.

In one embodiment, one of R₁₃, R₁₄, R₁₅, and R₁₆ is —H, (C₁-C₆)-alkyl, halogen, —CF₃, or —OR₂₅.

In one embodiment, R₁, R₂, R₃, R₆, R₇, R₈, R₉, R₁₀, R₁₁, R₁₂, R₁₃, R₁₄, R₁₅, and R₁₆ are each hydrogen.

In one embodiment, R₁, R₂, R₃, R₄, R₇, R₉, R₁₀, R₁₁, and R₁₂ are each hydrogen.

In one embodiment, R₁, R₂, R₃, R₄, R₇, R₈, R₁₀, R₁₁, and R₁₂ are each hydrogen.

In one embodiment, R₁, R₂, R₃, R₄, R₇, R₈, R₉, R₁₁, and R₁₂ are each hydrogen.

In one embodiment, R₁, R₂, R₃, R₄, R₇, R₈, R₉, R₁₀, and R₁₂ are each hydrogen.

In one embodiment, R₁, R₂, R₃, R₄, R₇, R₈, R₉, R₁₀, and R₁₁ are each hydrogen.

In one embodiment, R₁, R₂, R₃, R₄, R₇, R₈, and R₁₁ are each hydrogen.

In one embodiment, R₁, R₂, R₃, R₄, R₇, R₈, R₉ and R₁₁ are each hydrogen.

In one embodiment, R₁, R₂, R₃, R₄, R₅, R₆, R₇, R₈, R₉, and R₁₂ are each hydrogen.

In another embodiment, R₁₃, R₁₄, R₁₅, and R₁₆ are each hydrogen.

In one embodiment, R₃, R₆, R₇, R₈, R₉, R₁₂, R₁₃, R₁₄, R₁₅, and R₁₆ are each hydrogen.

In one embodiment, R₁, R₂, R₃, R₄, R₆, R₇, R₈, and R₁₁, are each hydrogen.

In one embodiment, R₁, R₂, R₃, R₄, R₆, R₇, R₈, R₁₁, R₁₃, R₁₄, R₁₅, and R₁₆ are each hydrogen.

In one embodiment, R₁, R₂, R₃, R₄, R₆, R₇, R₈, R₁₀, R₁₁, R₁₂, R₁₃, R₁₄, R₁₅, and R₁₆ are each hydrogen.

In one embodiment, R₁, R₂, R₃, R₄, R₆, R₇, R₈, R₉, R₁₀, R₁₁ and R₁₂ are each hydrogen.

In one embodiment, R₁, R₂, R₃, R₄, R₆, R₇, R₈, R₉, R₁₀, R₁₁, R₁₂ R₁₃, R₁₄, R₁₅, and R₁₆ are each hydrogen.

In one embodiment, R₁ is —H, —CF₃ or (C₁-C₆)-alkyl; R₄ and R₅ are each —H, halogen, —OR₂₅, or —CF₃; R₇, R₈, R₉, R₁₀, R₁₁, and R₁₂ are each —H, halogen, -alkyl, —OR₂₅, —CF₃, or —NO₂; and R₁₆ is —H or —CH₃.

In one embodiment, any one of R₁, R₂, R₃, R₄, R₅, and R₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN.

In one embodiment, any one of R₁, R₂, R₃, R₄, R₅, and R₆ is —H, (C₁-C₆)-alkyl, —OR₂₅, halogen, —CF₃, —NO₂, or —CN; and any two of R₇, R₈, R₉, R₁₀, R₁₁, and R₁₂ are each independently —H, (C₁-C₆)-alkyl, —OR₂₅, halogen, —CF₃.

In one embodiment, any one of R₁, R₂, R₃, R₄, R₅, and R₆ is —H, (C₁-C₆)-alkyl, —OR₂₅, halogen, —CF₃, —NO₂, or —CN; and any three of R₇, R₈, R₉, R₁₀, R₁₁, and R₁₂ are each independently —H, (C₁-C₆)-alkyl, —OR₂₅, halogen, —CF₃.

In one embodiment, any one of R₁, R₂, R₃, R₄, R₅, and R₆ is —H, (C₁-C₆)-alkyl, —OR₂₅, halogen or —CF₃; and any one of R₁₃, R₁₄, R₁₅, and R₁₆ is —H, (C₁-C₆)-alkyl, —OR₂₅, halogen or —CF₃.

In one embodiment, any one of R₇, R₈, R₉, R₁₀, R₁₁, and R₁₂ is —H, C₁-C₆)-alkyl, —OR₂₅, halogen, —CF₃, —NO₂, or —CN; and any one of R₁₃, R₁₄, R₁₅, and R₁₆ is —H, (C₁-C₆)-alkyl, —OR₂₅, halogen, —CF₃.

In one embodiment, R₄ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN.

In one embodiment, R₄ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any two of R₇, R₈, R₉, R₁₀, R₁₁, and R₁₂ are each independently —H, (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₅ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, -OR₂₅, halogen, or —CF₃, —NO₂, or —CN. In one embodiment, R₅ is —OR₂₅; any one of R₁₃, R₁₄, R₁₅, and R₁₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN.

In one embodiment, R₅ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any two of R₇, R₈, R₉, R₁₀, R₁₁, and R₁₂ are each independently —H, (C₁-C₆)-alkyl, —OR₂₅, halogen, or —CH₃, —NO₂, or —CN; wherein the any two of R₇, R₈, R₉, R₁₀, R₁₁, and R₁₂ can be either on the same ring of the quinoline or on different rings.

In one embodiment, R₅ is —OR₂₅; R₉ is halogen; any one of R₁₃, R₁₄, R₁₅, and R₁₆ is —H, (C₁-C₆)-alkyl, —OR₂₅, halogen or —CF₃; and any two of R₇, R₈, R₁₀, R₁₁, and R₁₂ are each independently —OR₂₅, halogen, or —CF₃; wherein the any two of R₇, R₈, R₉, R₁₀, R₁₁, and R₁₂ can be either on the same ring of the quinoline or on different rings.

In one embodiment, R₁ is —H or (C₁-C₆)-alkyl; R₂, R₈, and R₉ are each —H or halogen; R₄ is —H, halogen, —OR₂₅, or —CF₃; R₅ is —H, halogen, or —OR₂₅; and R₃, R₆, R₇, R₁₂, R₁₃, R₁₄, R₁₅, R₁₆, Rₐ and R_{b} are each hydrogen.

In one embodiment, R₁ is —H or —CH₃; R₂, R₈, and R₉ are each —H or F; R₄ is —H, F, —OCH₃, or —CF₃; R₅ is —H, F, or —OCH₃; and R₃, R₆, R₇, R₁₀, R₁₁, R₁₂, R₁₃, R₁₄, R₁₅, R₁₆, Rₐ and R_{b} are each hydrogen.

In one embodiment, R₂₅ is (C₁-C₆)-haloalkyl.

In another embodiment, R₂₅ is (C₁-C₆)-fluoroalkyl.

In one embodiment, R₂₅ is (C₁-C₆)-alkyl. In one embodiment, R₂₅ is —CH₃.

In some embodiments, the pharmacological agent is a compound or pharmaceutically acceptable salt of a compound of Formula II,

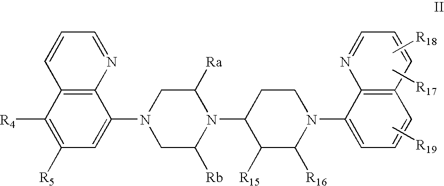

wherein Rₐ, R_{b}, R₄, R₅, R₁₅, R₁₆, and R₂₅ are defined as above for Formula (I), and

R₁₇, R₁₉, and R₁₉ are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, —C(O), —COR₂₅, —CO₂R₂₅, —NR₂₅CO₂R_{25, —NR}₂₅COR₂₅, —NR₂₅CON(R₂₅)₂, or —CON(R₂₅)₂.

In one embodiment, in the compound of Formula II, above:

R₄ and R₅ are each independently —H, —OR₂₅, halogen, or (C₁-C₆)-alkyl; R₁₅ and R₁₆ are each independently —H or —CH₃; and R₁₇, R₁₈, and R₁₉ are each independently —H, —OR₂₅, halogen, (C₁-C₆)-alkyl, —CF₃, —NO₂, —CN. In one embodiment, R₄ and R₅ are each independently —H, —OCH₃, F, or —CH₃; R₁₅ and R₁₆ are each independently —H or —CH₃; and R₁₇, R₁₈, and R₁₉ are each independently —H, —OCH₃, —F, —CH₃, —CF₃, —NO₂, —CN, or —Br.

In one embodiment, R₄ and R₅ are each independently —H, or —OR₂₅; R₁₅ and R₁₆ are each independently —H or —CH₃; and R₁₇, R₁₈, and R₁₉ are each independently —H, —OR₂₅, halogen, (C₁-C₆)-alkyl, or —CF₃. In one embodiment, R₄ and R₅ are each independently —H, or —OR₂₅; Rₐ, R_{b}, R₁₅ and R₁₆ are hydrogen; and R₁₇, R₁₈, and R₁₉ are each independently —H, —OR₂₅, halogen, (C₁-C₆)-alkyl, or —CF₃.

In another embodiment, R₁₉ is in the para position relative to the nitrogen of the piperidine.

In one embodiment, R₁₇ and R₁₈ are located at positions 2 and 4 of the quinoline ring (i.e., at the ortho and para positions relative to the nitrogen of the quinoline ring).

In one embodiment, R₅ is —H, (C₁-C₆)-alkyl, —OR₂₅, halogen, or —CF₃.

In another embodiment, R₅ is —H, (C₁-C₆)-alkyl, —OR₂₅, halogen, or —CF₃ and one of R₁₅ and R₁₆ is —H, (C₁-C₆)-alkyl, —OR₂₅, or halogen.

In yet another embodiment, R₅ is —H, (C₁-C₆)-alkyl, —OR₂₅, halogen, or —CF₃; R₁₅ is —H, (C₁-C₆)-alkyl, —OR₂₅, or halogen, and R₄ and R₁₆ are each hydrogen.

In one embodiment, R₅ is —H, (C₁-C₆)-alkyl, —OR₂₅, halogen, or —CF₃; R₁₆ is —H, (C₁-C₆)-alkyl, —OR₂₅, or halogen, and R₄ and R₁₅ are each hydrogen.

In one embodiment, R₅ is —H, (C₁-C₆)-alkyl, —OR₂₅, halogen or —CF₃ and R₄, R₁₅, R₁₆, R₁₇, R₁₈ and R₁₉ are each hydrogen.

In one embodiment, R₅ is —H, (C₁-C₆)-alkyl, —OR₂₅, halogen, or —CF₃ and one of R₁₇, R₁₈ and R₁₉ is —H, (C₁-C₆)-alkyl, —OR₂₅, halogen, or —CF₃. In another embodiment, R₅ is —H, (C₁-C₆)-alkyl, —OR₂₅, halogen, or —CF₃; one of R₁₇, R₁₈ and R₁₉ is —H, (C₁-C₆)-alkyl, —OR₂₅, halogen, or —CF₃; and each remaining R group is hydrogen.

In one embodiment, R₅, R₁₇, R₁₈, and R₁₉ are each independently —H, (C₁-C₆)-alkyl, —OR₂₅, halogen or —CF₃ and R₄, R₁₅, and R₁₆ are each hydrogen. In one embodiment, R₅ is —H, —OR₂₅ or halogen; R₁₇ and R₁₈ are each independently —H, (C₁-C₆)-alkyl, —OR₂₅, halogen or —CF₃; R₁₉ is —H or halogen; and Rₐ, R_{b}, R₄, R₁₅, and R₁₆ are each hydrogen.

In one embodiment, R₄ is —H, (C₁-C₆)-alkyl, —OR₂₅, halogen, or —CF₃.

In another embodiment, R₄ is —H, (C₁-C₆)-alkyl, —OR₂₅, halogen, or —CF₃ and one of R₁₅, and R₁₆ is —H, (C₁-C₆)-alkyl, —OR₂₅, or halogen.

In a further embodiment, R₄ is —H, (C₁-C₆)-alkyl, —OR₂₅, halogen, or —CF₃; one of R₁₅ and R₁₆ is —H, (C₁-C₆)-alkyl, —OR₂₅, or halogen; and R₁₇, R₁₈ and R₁₉ are each hydrogen.

In yet another embodiment, R₄ is —H, (C₁-C₆)-alkyl, —OR₂₅, halogen, or —CF₃; R₁₅ is —H, (C₁-C₆)-alkyl, —OR₂₅, or halogen, and R₅ and R₁₆ are each hydrogen.

In one embodiment, R₄ is —H, (C₁-C₆)-alkyl, —OR₂₅, halogen, or —CF₃; R₁₆ is —H, (C₁-C₆)-alkyl, —OR₂₅, or halogen, and R₅ and R₁₅ are each hydrogen.

In one embodiment, R₄ is —H, (C₁-C₆)-alkyl, —OR₂₅, halogen or —CF₃ and R₅, R₁₅, R₁₆, R₁₇, R₁₈ and R₁₉ are each hydrogen.

In one embodiment, R₅ is —H, (C₁-C₆)-alkyl, —OR₂₅, halogen, or —CF₃ and one of R₁₇, R₁₈ and R₁₉ is —H, (C₁-C₆)-alkyl, —OR₂₅, halogen, or —CF₃.

In one embodiment, R₅ is —H, (C₁-C₆)-alkyl, —OR₂₅, halogen, or —CF₃ and two of R₁₇, R₁₈ and R₁₉ are each independently —H, (C₁-C₆)-alkyl, —OR₂₅, halogen, or —CF₃.

In one embodiment, R₅, R₁₇, R₁₈ and R₁₉ are each independently —H, (C₁-C₆)-alkyl, —OR₂₅, halogen, or —CF₃.

In another embodiment, R₅ is —H, (C₁-C₆)-alkyl, —OR₂₅, halogen, or —CF₃ and one of R₁₇, R₁₈ and R₁₉ is —H, (C₁-C₆)-alkyl, —OR₂₅, halogen, or —CF₃; and each remaining R group is hydrogen.

In another embodiment, R₅ is —H, (C₁-C₆)-alkyl, —OR₂₅, halogen, or —CF₃ and two of R₁₇, R₁₈ and R₁₉ are each independently —H, (C₁-C₆)-alkyl, —OR₂₅, halogen, or —CF₃; and each remaining R group is hydrogen.

In one embodiment, R₅, R₁₇, R₁₈ and R₁₉ are each independently —H, (C₁-C₆)-alkyl, —OR₂₅, halogen, or —CF₃ and each remaining R group is hydrogen.

In a further embodiment, R₅ is —H, (C₁-C₆)-alkyl, —OR₂₅, halogen, or —CF₃; one of R₁₇, R₁₈ and R₁₉ is —H, (C₁-C₆)-alkyl, —OR₂₅, halogen, or —CF₃; one of R₁₅ and R₁₆ is —H, (C₁-C₆) alkyl, —OR₂₅, or halogen; and each remaining R group is hydrogen.

In one embodiment, R₅ is —H, (C₁-C₆)-alkyl, —OR₂₅, halogen, or —CF₃; and any two of R₁₇, R₁₈ and R₁₉ are each independently —H, (C₁-C₆)-alkyl, —OR₂₅, halogen, or —CF₃; and one of R₁₅, and R₁₆ is —H, (C₁-C₆)-alkyl, —OR₂₅, halogen, or —CF₃.

In one embodiment, R₅ is —H, (C₁-C₆)-alkyl, —OR₂₅, halogen, or —CF₃; R₁₇, R₁₈ and R₁₉ are each independently —H, (C₁-C₆)-alkyl, —OR₂₅, halogen, or —CF₃; and one of R₁₅, and R₁₆ is —H, (C₁-C₆)-alkyl, —OR₂₅, halogen, or —CF₃

In one embodiment, R₄ is —H, (C₁-C₆)-alkyl, —OR₂₅, halogen, or —CF₃ and one of R₁₇, R₁₈ and R₁₉ is —H, (C₁-C₆)-alkyl, —OR₂₅, halogen, or —CF₃.

In another embodiment, R₄ is —H, (C₁-C₆)-alkyl, —OR₂₅, halogen, or —CF₃ and one of R₁₇, R₁₈ and R₁₉ is —H, (C₁-C₆)-alkyl, —OR₂₅, halogen, or —CF₃; and each remaining R group is hydrogen.

In a further embodiment, R₄ is —H, (C₁-C₆)-alkyl, —OR₂₅, halogen, or —CF₃; one of R₁₇, R₁₈ and R₁₉ is —H, (C₁-C₆)-alkyl, —OR₂₅, halogen, or —CF₃; one of R₁₅ and R₁₆ is —H, (C₁-C₆)-alkyl, —OR₂₅, or halogen; and each remaining R group is hydrogen.

In a further embodiment, R₄ is —H, (C₁-C₆)-alkyl, —OR₂₅, halogen, or —CF₃; and any two of R₁₇, R₁₈ and R₁₉ are each independently —H, (C₁-C₆)-alkyl, —OR₂₅, halogen, or —CF₃ . In one embodiment, R₄, R₁₇, R₁₈ and R₁₉ are each independently —H, (C₁-C₆)-alkyl, —OR₂₅, halogen, or —CF₃ and each remaining R group is hydrogen.

In one embodiment, one of R₁₅ and R₁₆ is —H, (C₁-C₆)-alkyl, halogen, —CF₃, or —OR₂₅. In a further embodiment, one of R₁₅ and R₁₆ is —H, (C₁-C₆)-alkyl, halogen, —CF₃, or —OR₂₅; R₅ is —H, (C₁-C₆)-alkyl, —OR₂₅, halogen, or —CF₃; and each remaining R group is hydrogen.

In a further embodiment, one of R₁₅ and R₁₆ is —H, (C₁-C₆)-alkyl, halogen, —CF₃, or —OR₂₅; R₄ is —H, (C₁-C₆)-alkyl, —OR₂₅, halogen, or —CF₃; and each remaining R group is hydrogen.

In one embodiment, R₄, R₁₅, R₁₆, R₁₇, R₁₈ and R₁₉ are each hydrogen.

In one embodiment, R₄, R₁₅, R₁₆, R₁₇, and R₁₈ are each hydrogen.

In one embodiment, R₄, R₁₅, and R₁₆ are each hydrogen.

In one embodiment, R₅, R₁₅, R₁₆, R₁₇, R₁₈ and R₁₉ are each hydrogen.

In one embodiment, R₅, R₁₅, R₁₆, R₁₇, and R₁₈ are each hydrogen.

In one embodiment, R₄, R₁₅, R₁₆, and R₁₉ are each hydrogen.

In one embodiment, R₅, R₁₅, and R₁₆ are each hydrogen.

In one embodiment, R₅ is —H, —OR₂₅ or halogen; R₄, R₁₅, R₁₆, R₁₇, and R₁₈ are each hydrogen; and R₁₉ is —H or halogen.

In one embodiment, R₅ is —H, —OCH₃ or F; R₄, R₁₅, R₁₆, R₁₇, and R₁₈ are each hydrogen; and R₁₉ is —H or F.

In one embodiment, R₅ is —H, —OCH₃ or F; R₄, R₁₅, and R₁₆ are each hydrogen; and one of R₁₈ or R₁₉ is —H or F. In one embodiment, R₅ is —H, —OCH₃ or F; R₄, R₁₅, R₁₆ and R₁₇ are each hydrogen; and R₁₈ and R₁₉ are each independently —H, —CH₃ or halogen.

In one embodiment, R₄ is hydrogen, R₅ is —H, —OR₂₅; and R₁₇, R₁₈, and R₁₉ are each independently —H, —OR₂₅, halogen, (C₁-C₆)-alkyl, or —CF₃. In one embodiment, R₄ is hydrogen, R₅ is —OR₂₅; R₁₇, and R₁₈ are each independently —H, —OR₂₅, halogen, (C₁-C₆)-alkyl, or —CF₃; and R₁₉ is —H or halogen.

In one embodiment, R₅ is —H, —OR₂₅ or halogen; R₁₇ and R₁₈ are each independently —H, (C₁-C₆)-alkyl, —OR₂₅, halogen or —CF₃; R₁₉ is H or halogen; and Rₐ, R_{b}, R₄, R₁₅, and R₁₆ are each hydrogen.

In one embodiment, R₅ is —H, —OCH₃ or F; R₁₇ is —H, —OCH₃; R₁₈ is —H, —CF₃; R₁₉ is —H, F; and Rₐ, R_{b}, R₄, R₁₅, and R₁₆ are each hydrogen.

In one embodiment, R₄ is —H, —OR₂₅ or halogen; R₅, R₁₅, R₁₆, R₁₇ and R₁₈ are each hydrogen; and R₁₉ is —H or halogen. In one embodiment, R₅ is —H, —OCH₃ or F; R₄, R₁₅, R₁₆ and R₁₉ are each hydrogen; and R₁₇ and R₁₈ are each —H, —CH₃ or halogen.

In one embodiment, R₄ is —H, —OCH₃ or F; R₅, R₁₅, R₁₆, R₁₇ and R₁₈ are each hydrogen; and R₁₉ is —H or F.

In one embodiment, R₄ is —H, —OCH₃ or F; R₅, R₁₅, and R₁₆ are each hydrogen; and one of R₁₈ or R₁₉ is —H or F. In one embodiment, R₄ is —H, —OCH₃ or F; R₅, R₁₅, R₁₆ and R₁₇ are each hydrogen; and R₁₈ and R₁₉ are each —H, —CH₃ or halogen. In one embodiment, R₄ is —H, —OCH₃ or F; R₄, R₁₅, R₁₆ and R₁₉ are each hydrogen; and R₁₇ and R₁₈ are each —H, —CH₃ or halogen.

In some embodiments, the pharmacological agent is one of the illustrative examples of compounds of Formula I and Formula II set forth below:

6-methoxy-8-[4-(1-quinolin-8-yl-piperidin-4-yl)-piperazin-1-yl]-quinoline;

6-fluoro-8-[4-(1-quinolin-8-yl-piperidin-4-yl)-piperazin-1-yl]-quinoline;

5-fluoro-8-[4-(1-quinolin-8-yl-piperidin-4-yl)-piperazin-1-yl]-quinoline;

7-fluoro-8-(4-(4-(6-methoxyquinolin-8-yl)piperazin-1-yl)piperidin-1-yl)quinoline;

6-fluoro-8-{4-[1-(8-fluoroquinolin-7-yl)piperidin-4-yl]piperazin-1-yl}quinoline;

3-trifluoromethyl-8-(4-(4-(6-methoxyquinolin-8-yl)piperazin-1-yl)piperidin-1-yl)quinoline;

6-methoxy-8-(4-(1-(quinolin-8-ylmethyl)piperidin-4-yl)piperazin-1-yl)quinoline;

5-fluoro-4-methoxy-8-(4-(4-(6-methoxyquinolin-8-yl)piperazin-1-yl)piperidin-1-yl)-2-(trifluoromethyl)quinoline;

5-fluoro-8-(4-(4-(6-methoxyquinolin-8-yl)piperazin-1-yl)piperidin-1-yl)quinoline;

8-[4-(1-quinolin-8-yl-piperidin-4-yl)-piperazin-1-yl]-quinoline;

6-chloro-8-[4-(4-(6-chloro)-quinolin-8-yl-piperidin-1-yl)-piperazin-1-yl]-quinoline;

6-fluoro-8-[4-(4-(6-chloro)-quinolin-8-yl-piperidin-1-yl)-piperazin-1-yl]-quinoline;

5-chloro-8-[4-(1-quinolin-8-yl-piperidin-4-yl)-piperazin-1-yl]-quinoline;

2-methyl-8-[4-(1-quinolin-8-yl-piperidin-4-yl)-piperazin-1-yl]-quinoline;

6-chloro-8-[4-(1-quinolin-8-yl-piperidin-4-yl)-piperazin-1-yl]-quinoline;

8-[4-(1-quinolin-8-yl-piperidin-4-yl)-piperazin-1-yl]-5-trifluoromethyl-quinoline;

5-methoxy-8-[4-(1-quinolin-8-yl-piperidin-4-yl)-piperazin-1-yl]-quinoline;

5-fluoro-8-[4-(4-quinolin-8-yl-piperazin-1-yl)-piperidin-1-yl]-quinoline;

6-methoxy-8-[4-(2-methylquinolin-8-yl-piperidin-4-yl)-piperazin-1-yl]-quinoline;

6-fluoro-8-(4-(1-(2-methylquinolin-8-yl)piperidin-4-yl)piperazin-1-yl)quinoline;

6-methoxy-8-[4-(3-methylquinolin-8-yl-piperidin-4-yl)-piperazin-1-yl]-quinoline;

6-methoxy-8-(4-(1-(4-methylquinolin-8-yl)piperidin-4-yl)piperazin-1-yl)quinoline;

6-methoxy-8-(4-(1-(2,4-dimethylquinolin-8-yl)piperidin-4-yl)piperazin-1-yl)quinoline;

6-methoxy-8-(4-(1-(2,4-dimethyl-5-fluoroquinolin-8-yl)piperidin-4-yl)piperazin-1-yl)quinoline;

6-methoxy-8-(4-(1-(2-(trifluoromethyl)quinolin-8-yl)piperidin-4-yl)piperazin-1-yl)quinoline;

6-fluoro-8-(4-(1-(5-fluoroquinolin-8-yl)piperidin-4-yl)piperazin-1-yl)quinoline;

6-methoxy-8-(4-(1-(6-bromoquinolin-8-yl)piperidin-4-yl)piperazin-1-yl)quinoline;

6-methoxy-8-(4-(1-(6-fluoroquinolin-8-yl)piperidin-4-yl)piperazin-1-yl)quinoline;

6-fluoro-8-(4-(1-(7-fluoroquinolin-8-yl)piperidin-4-yl)piperazin-1-yl)quinoline;

6-methoxy-8-{4-[1-(8-fluoroquinolin-7-yl)piperidin-4-yl]piperazin-1-yl}quinoline;

6-methoxy-8-{4-[1-(2-trifluoromethyl-4-methoxyquinolin-7-yl)piperidin-4-yl]piperazin-1-yl}quinoline;

6-methoxy-8-(4-(1-(2-trifluoromethyl-4-methoxyquinolin-8-yl)piperidin-4-yl)piperazin-1-yl)quinoline;

5-fluoro-8-(4-(4-(6-methoxyquinolin-8-yl)piperazin-1-yl)piperidin-1-yl)-2-trifluoromethylquinoline;

5-fluoro-8-(4-(4-(6-methoxyquinolin-8-yl)piperazin-1-yl)piperidin-1-yl)-3-trifluoromethylquinoline;

5-fluoro-8-(4-(4-(6-methoxyquinolin-8-yl)piperazin-1-yl)piperidin-1-yl)-4-trifluoromethylquinoline;

2,5-difluoro-8-(4-(4-(6-methoxyquinolin-8-yl)piperazin-1-yl)piperidin-1-yl)quinoline;

3,5-difluoro-8-(4-(4-(6-methoxyquinolin-8-yl)piperazin-1-yl)piperidin-1-yl)quinoline;

4,5-difluoro-8-(4-(4-(6-methoxyquinolin-8-yl)piperazin-1-yl)piperidin-1-yl)quinoline;

and pharmaceutically acceptable salts thereof.

In one embodiment, the pharmacological agent is 5-fluoro-8-{4-[4-(6-methoxyquinolin-8-yl)piperazin-1-yl]piperidin-1-yl}quinoline or a pharmaceutically acceptable salt thereof. In one embodiment, the pharmacological agent is 5-fluoro-8-{4-[4-(6-methoxyquinolin-8-yl)piperazin-1-yl]piperidin-1-yl}quinoline succinate. In one embodiment, the pharmacological agent is 5-fluoro-8-{4-[4-(6-methoxyquinolin-8-yl)piperazin-1-yl]piperidin-1-yl}quinoline trisuccinate.

The compounds and pharmaceutically acceptable salts of compounds described herein can contain an asymmetric carbon atom and some of the compounds or pharmaceutically acceptable salts of compounds can contain one or more asymmetric centers, and can thus give rise to optical isomers and diastereomers. While described without respect to stereochemistry herein, the present invention includes such optical isomers and diastereomers, as well as racemic and resolved, enantiomerically pure R and S stereoisomers, and also other mixtures of the R and S stereoisomers and pharmaceutically acceptable salts thereof. Where a stereoisomer is preferred, it can in some embodiments be provided substantially free of its corresponding opposite enantiomer.

In addition, the compounds and pharmaceutically acceptable salts of compounds described herein can exist as polymorphs. Such polymorphs can be transient or isolatable as a stable product. Examples of some polymorphs of the compounds described herein are included in U.S. patent application Ser. No. 11/811,150, entitled “6-Methoxy-8-[4-(1-(5-Fluoro)-Quinolin-8-yl-Piperidin-4-yl)-Piperazin-1-yl]-Quinoline Hydrochloric Acid Salts”, filed Jun. 8, 2007, and U.S. patent application Ser. No. 11/811,022, entitled “Succinate Salts Of 6-Methoxy-8-[4-(1-(5-Fluoro)-Quinolin-8-Yl-Piperidin-4-Yl)-Piperazin-1-Yl]-Quinoline And Crystalline Forms Thereof”, filed Jun. 8, 2007, each of which is incorporated by reference in its entirety. For example, in some embodiments, the compounds or pharmaceutically acceptable salts of the compounds described herein are Forms A, B, C, or D of 6-methoxy-8-[4-(1-(5-fluoro)-quinolin-8-yl-piperidin-4-yl)-piperazin-1-yl]-quinoline trisuccinate.

Prodrugs of the compounds or pharmaceutically acceptable salts of compounds are also within the scope of the present invention.

The compounds described herein are prepared according to methods described in US 2007/0027160, entitled “Piperazine-Piperidine Antagonists and Agonists of the 5-HT_{1A} Receptor,” and co-pending U.S. patent application Ser. No. 11/811,328, filed Jun. 7, 2007, entitled “Process For Synthesizing Piperazine-Piperidine Compounds,” each of which is herein incorporated by reference in its entirety.

In some embodiments, the pharmacological agent is micronized. Without being bound to a particular theory, it is thought that micronization facilitates dissolution. In some embodiments, approximately 50% of the pharmacological agent is less than about 10 μm, or approximately 40% of the pharmacological agent is less than about 10 μm, or approximately 30% of the pharmacological agent is less than about 10 μm, or approximately 25% of the pharmacological agent is less than about 10 μm. In some embodiments, approximately 90% of the pharmacological agent is less than about 25 μm, or approximately 80% of the pharmacological agent is less than about 25 μm, or approximately 75% of the pharmacological agent is less than about 25 μm. Particle size distribution is determined on a weight basis using techniques known to those of skill in the art, such as dynamic or static light scattering or microscopy.

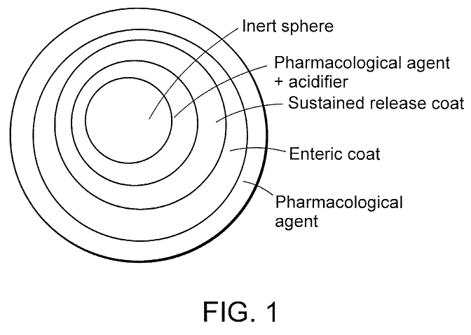

In certain embodiments, the present invention provides a controlled-release bead prepared by applying one or more coatings including the pharmacological agent, one or more sustained-release coat, and one or more enteric coat. In one embodiment, as shown in FIG. 1, the controlled-release bead provides an outermost layer including a pharmacological agent that provides immediate release of the pharmacological agent in the stomach (i.e., where the agent has high solubility); an enteric layer that delays the further release of the pharmacological agent until the composition reaches the pH of the small intestine; a sustained release layer that slows and controls the release of the pharmacological agent by a diffusion process; and an inner layer including an additional amount of the pharmacological agent further including a pH modifying material, such as an acidifier, where the pH modifying material is slowly released with the pharmacological agent to effect a lower microenvironmental pH, thereby improving dissolution in a region of otherwise inhospitable pH. The multilayer controlled-release beads described herein have the benefit of a simplified manufacturing process by applying all the coating ingredients in a single unit operation. In certain embodiments, the outermost layer may also include a pharmacological agent and a sustained release coat.

The acidifier alters the microenvironmental pH to improve solubility of the pharmacological agent. For example, the acidifier adjusts and stabilizes the pH of the area immediately surrounding the pharmaceutical composition, providing for improved drug release. Thus, the acidifier is chosen based on its ability to maintain a particular microenvironmental pH. For example, in some embodiments, the acidifier is chosen to create a microenvironmental pH in the range associated with a peak or maximum in the solubility profile of a pharmacological agent. Exemplary acidifiers include, without limitation, one or more of citric acid, ascorbic acid, glutamic acid, tartaric acid, succinic acid, malic acid, erythorbic acid, propionic acid, lactic acid, oleic acid, fumaric acid, benzoic acid, and alginic acid. In some embodiments, the acidifier is present in the pharmaceutical composition at a level from about 0.01% by weight to about 20% by weight of the pharmaceutical composition. In some embodiments, the acidifier is present at a level from about 0.5% to about 10% by weight, for example at about 1%, about 2%, about 3%, about 4%, about 5%, about 6%, about 7%, about 8%, or about 9%. In some embodiments, the acidifier is present at a level from about 1% to about 5% by weight, for example at about 1.5%, about 2.5%, about 3.5%, about 4.5%. In one embodiment, the acidifier is citric acid. At a pH approximating intestinal pH (i.e., pH of about 5.5 or higher, e.g., ˜6.8), the citric acid creates a localized acidic environment in and around the pharmaceutical composition as it releases the pharmacological agent. In certain embodiments, the acidifier improves the in vitro dissolution of the pharmacological agent at a pH level corresponding to the pH of the lower gastrointestinal tract.

In certain embodiments, the present invention provides a controlled-release bead comprising:

(i) a core unit of a substantially water-soluble or water-swellable inert material;

(ii) a first layer on the core unit comprising a pharmacological agent, an acidifier and optionally a binder;

(iii) a second layer of sustained-release coat covering the first layer;

(iv) a third layer of enteric coat on the second layer; and

(v) optionally, an outermost layer comprising the pharmacological agent and optionally a binder.

In certain other embodiments, the present invention provides a controlled-release bead comprising:

(i) a core unit comprising a mixture of a substantially water-soluble or water-swellable inert material, a pharmacological agent, an acidifier and optionally a binder;

(ii) a first layer of sustained-release coat on the core unit;

(iii) a second layer of enteric coat covering the first layer; and

(iv) optionally, an outermost layer comprising the pharmacological agent and optionally a binder.

In certain embodiments, the sustained-release coat is effective for controlled release of the pharmacological agent contained in the first layer or the core unit, the enteric coat is effective for delaying the onset of the release of the pharmacological agent contained in the first layer or the core unit, and the outermost layer is effective for immediate release of the pharmacological agent contained in the outermost layer.

In certain embodiments, the outermost layer is present and the ratio between the pharmacological agent contained in the outermost layer to that contained in the first layer or the core unit is from about 15% to about 40% w/w. In certain embodiments, the ratio is from about 20% to about 35% w/w. In certain other embodiments, the ratio is from about 25% to about 30% w/w.

In certain embodiments, The controlled-release bead comprises:

a) the water-soluble or water-swellable inert material comprises from about 60% to about 90% by weight of the bead;

b) the pharmacological agent comprises from about 1% to about 25% by weight of the bead;

c) the acidifier comprises from about 0.5% to about 10% by weight of the bead;

d) the sustained-release coat comprises from about 1% to about 20% by weight of the bead;

e) the binder comprises from about 0.1% to about 5% by weight of the bead; and

f) the enteric coat comprises from about 0.5% to about 20% by weight of the bead, in which the enteric coat contains from about 0.5% to about 15% of an enteric coating polymer or copolymer by weight of the bead, from about 0.01% to about 2% of a pH adjustment agent by weight of the bead, from about 0.1% to about 5% of a glidant by weight of the bead, from about 0.1% to about 3% of a plasticizer by weight of the bead, and from about 0.01% to about 2% of a surfactant by weight of the bead.

In certain embodiments, the pharmacological agent comprises from about 1% to about 10% by weight of the bead; the acidifier comprises from about 1% to about 5% by weight of the bead; the sustained-release coat comprises from about 5% to about 15% by weight of the bead; and the enteric coat comprises from about 1% to about 15% by weight of the bead.

In some embodiments, the controlled-release bead contains one or more of a sustained release coat, an enteric coat, and a pharmacological agent coat. In some embodiments, the controlled-release bead contains a sustained release coat over the core containing the pharmacological agent. Similarly, in some embodiments, the controlled-release bead contains an enteric coat over the core. In some embodiments, the controlled-release bead contains both a sustained release coat and an enteric coat. When both coats are present, they can be applied in any order, depending on the drug release profile desired. For example, in one embodiment, the sustained release coat is interposed between the core and the enteric coat. The enteric coat ensures that the pharmacological agent in the core does not release quickly in the stomach, which has a relatively low pH. The sustained release layer is applied as the second layer, adjacent to the core, to sustain release of the agent at the higher pH of the small intestine.

In certain embodiments, the controlled-release bead provides a dissolution profile characterized in that about 15% to about 35% by weight of the pharmacological agent is released after about 2 hours and about 45% to about 65% by weight of the pharmacological agent is released after about 8 hours, in Apparatus 2 described in the United States Pharmacopoeia (USP29-NF24, page 2673) having a rotation speed of 75 rpm, and a two stage dissolution medium containing (i) pH˜1 buffer for about two hours, and followed by (ii) pH˜6.8 buffer with 1% SLS (sodium lauryl sulfate) for about 6 additional hours.

In certain other embodiments, the controlled-release bead provides a dissolution profile characterized in that less than about 15% by weight of the pharmacological agent is released after about 2 hours and more than about 60% by weight of the pharmacological agent is released after about 8 hours, in Apparatus 2 described in the United States Pharmacopoeia (USP29-NF24, page 2673) having a rotation speed of 75 rpm, and a two stage dissolution medium containing (i) pH˜1 buffer for about two hours, and followed by (ii) pH˜6.8 buffer with 1% SLS (sodium lauryl sulfate) for about 6 additional hours.

In certain embodiments, the core unit is preferably a water-soluble or water-swellable inert material, and can be any such material that is conventionally used as cores or any other pharmaceutically acceptable water-soluble or water-swellable material made into beads or beads. Exemplary examples of a water-soluble or water-swellable inert material include, but are not limited to, spheres of sucrose, starch, Sugar Spheres NF, sucrose crystals, microcrystalline cellulose, lactose, and mixtures thereof.

In some embodiments, the pharmacological agent and the acidifier can be combined to form the core unit of the controlled-release bead, preferably with at least one pharmaceutically acceptable excipient. For example, the pharmacological agent and the acidifier can be combined with the water-soluble or water-swellable inert material to form the core unit of the controlled-release bead. In one embodiment, the pharmacological agent and the acidifier are combined to form a core or a mini-tablet, such that there is a uniform or near-uniform concentration of the pharmacological agent throughout the core. For example, in some embodiments, the pharmacological agent, the acidifier and the water-soluble or water-swellable inert material are compressed after mixing to form a sphere or mini-tablet. In other embodiments, the pharmacological agent and the acidifier are extruded and/or spheronized with appropriate excipients to form a mini-tablet, which can then be coated. In other embodiments, wet granulation, dry granulation or dry blending can be used to prepare a mini-tablet.

In certain other embodiments, the pharmacological agent and the acidifier are applied to the surface of the water-soluble or water-swellable inert material, such as a nonpareil bead, a sugar sphere, or a cellulose sphere (e.g., microcrystalline cellulose). Any art-recognized method for applying pharmaceutical coatings may be used to apply the pharmacological agent/acidifier mixture to the core (as well as other coatings described herein). Examples of such methods include, without limitation, spraying a suspension or solution, powder layering, and compression coating. The pharmacological agent and the acidifier are combined by any art-recognized method for combining pharmaceutical ingredients. Exemplary methods include, without limitation, solutions, suspensions or dispersions, wet granulation, dry granulation, and dry blending.

In a further embodiment, when the pharmaceutical composition includes an inert core material, the acidifier can be incorporated into the material itself. For example, in some embodiments, the inert material is manufactured by an extrusion and/or spheronization, beadization, or marumerization process, to incorporate the acidifier, as well as the other inert materials. The pharmacological agent is then applied to the surface of the core, followed by the sustained release coat and/or the enteric coat and the optional pharmacological agent coat.

In some embodiments, the core unit or layer containing the pharmacological agent also contains other excipients, including, without limitation, one or more of the binders described herein. Nonlimiting examples of such excipients include microcrystalline cellulose, cellulose, carboxymethyl cellulose, methylcellulose, hydroxypropyl methylcellulose, lactose, polyvinylpyrrolidone, mannitol, dicalcium phosphate, and maltodextrin, starch. In some embodiments, the binder comprises from about 0.1% to about 5% by weight of the bead.

In certain embodiments, the mixture containing the pharmacological agent and the acidifier are applied to the core unit (e.g., inert sphere) by any suitable method known to those of skill in the art. For example, in some embodiments, the mixture is applied by spray drying. In other embodiments, the mixture is applied through the use of a fluid-bed coater, a coating pan, powder layering or compression coating. In one embodiment, the mixture is applied by film coating in a fluid bed coater with a column insert.

The sustained release coat typically, although not necessarily, includes a polymeric material as the sustained release component. Such materials include any polymer material suitable for pharmaceutical dosage forms that retard the release of drug substances from such dosage forms. Examples of suitable polymers for use as the sustained release coat can be found in Remington's Pharmaceutical Sciences, 18th Ed., Gennaro, ed., Mack Publishing Co., Easton, Pa., 1990, incorporated by reference herein in its entirety for all purposes. In some embodiments, the sustained release component includes, without limitation, one or more of ethylcelluloses, polyvinyl acetates, polymers or copolymers or acrylates or methacrylates, or cellulose acetate. In some embodiments, the sustained release component includes, without limitation, one or more of polymethacrylates, methacrylic acid-methacrylic acid ester copolymers, acrylate methacrylate copolymers, ethylacrylate/methylmethacrylate copolymers, cellulose acetate, ethylcellulose, high viscosity matrix forming hydroxypropyl methyl celluloses such as Methocel K4M, Methocel K15M, Methocel K100M, Methocel E4M, and low viscosity matrix forming hydroxypropyl methyl celluloses such as Methocel K100LV, Methocel E50LV, Methocel E5, Methocel E15LV. In some embodiments, the sustained release coat includes an ethylcellulose-based product, such as the commercially available Surelease™ aqueous ethylcellulose dispersion product (Colorcon, Inc.). For example, in some embodiments, the sustained release coat includes Surelease-E-7-19010, containing ethylcellulose and other ingredients including ammonium hydroxide Combinations of different coating materials may also be used as a sustained release coating. The sustained release coat initially can form a premix, solution, or suspension of any of the above coating materials.

In some embodiments, the sustained release coat is present in the composition in an amount ranging from about 0.01% to about 30% by weight of the pharmaceutical composition. In some embodiments, the sustained release component is present in the composition in an amount ranging from about 1% to about 20% by weight of the pharmaceutical composition. In some embodiments, the sustained release component is present in the composition in an amount ranging from about 1% to about 15% by weight of the pharmaceutical composition. In some embodiments, the sustained release component is present in the composition in an amount ranging from about 1% to about 10% by weight of the pharmaceutical composition. In some embodiments, the sustained release component is present in the composition in an amount ranging from about 5% to about 15% by weight of the pharmaceutical composition.

The enteric coat also typically, although not necessarily, includes a polymeric material as the enteric component. Enteric coating materials generally comprise polymers that are insoluble in gastric media and hydrate and dissolve in the upper intestine. This is typically dictated by the pH change of the gastrointestinal media and, generally, ionization of carboxylic acid groups on the polymer. Any coating should be applied to a sufficient thickness such that the entire coating does not dissolve in the gastrointestinal fluids at pH below about 5, but does dissolve at pH about 5 or 5.5 and above. The enteric coat also inhibits release of the acidifier (e.g., citric acid), such that its release coincides with the release of the pharmacological agent. It is expected that any anionic polymer exhibiting a pH-dependent solubility profile can be used as an enteric coating in the practice of the present invention to achieve delivery of the active to the lower gastrointestinal tract. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

In certain embodiments, the enteric coat comprises an enteric coating polymer or copolymer, an optional pH adjustment agent, an optional glidant, an optional plasticizer, an optional surfactant, and mixtures thereof.

Nonlimiting examples of suitable enteric coating polymer or copolymer include, but are not limited to: one or more of polymers and/or copolymers of acrylates or methacrylates, cellulose acetate phthalate, or hydroxypropylmethyl cellulose acetate phthalate. Other examples of enteric components include, without limitation, cellulosic polymers, such as cellulose acetate phthalate, diethyl phthalate, dibutyl phthalate, cellulose acetate trimellitate, hydroxypropylmethyl cellulose phthalate, hydroxypropylmethyl cellulose acetate succinate, hydroxypropylmethyl cellulose acetate succinate and carboxymethylcellulose sodium; acrylic acid polymers and copolymers, for example formed from acrylic acid, methacrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers, such as polyvinyl pyrrolidone (PVP), polyvinyl acetate, polyvinyl acetate phthalate, vinyl acetate crotonic acid copolymer, and ethylene-vinyl acetate copolymers, poly(methyle vinyl ether/maleic anhydride); zein and shellac (purified lac). Combinations of different coating materials may also be used as an enteric coat component. Prior to coating, the enteric coating or component can be in the form of a premix, solution or suspension including one of the enteric coating materials described herein.

The enteric coat provides for controlled release of the pharmacological agent, such that drug release can be accomplished at some generally predictable location in the lower intestinal tract below the point at which drug release would occur without the enteric coating. For example, the pharmaceutical compositions described herein provide optimized drug availability for absorption through slow release in the intestine. The enteric coat also prevents exposure of the pharmacologic agent and any carriers to the epithelial and mucosal tissue of the buccal cavity, pharynx, esophagus, and stomach, and to the enzymes associated with these tissues. The enteric coat therefore helps to protect the active agent and a patient's internal tissue from any adverse event prior to drug release at the desired site of delivery. Furthermore, the coated capsules of the present invention allow optimization of drug absorption, active agent protection, and safety. In some embodiments, multiple enteric coatings targeted to release the pharmacologic agent at various regions in the lower gastrointestinal tract are included in the composition, thereby enabling, in theory, even more effective and sustained improved delivery throughout the lower gastrointestinal tract.

In some embodiments, the enteric coating polymer or copolymer is an anionic polymer selected from the group consisting of a methacrylic acid copolymer, cellulose acetate phthalate, hydroxpropylmethylcellulose phthalate, polyvinyl acetate phthalate, shellac, hydroxpropylmethylcellulose acetate succinate, and carboxy-methylcellulose. In some embodiments, the enteric coating polymer or copolymer is a methacrylic acid copolymer. In some embodiments, the enteric coating polymer or copolymer is methyl methacrylate, ethyl methacrylate or mixtures thereof. In some embodiments, the enteric coating polymer or copolymer is Eudragit polymer.

The enteric coat is typically present in the composition in an amount ranging from about 0.01% to about 50% by weight of the pharmaceutical composition. In some embodiments, the enteric coat component is present in the composition in an amount ranging from about 0.5% to about 20% by weight of the pharmaceutical composition. In some embodiments, the enteric coat component is present in the composition in an amount ranging from about 1% to about 15% by weight of the pharmaceutical composition. In some embodiments, the enteric coat component is present in the composition in an amount ranging from about 1% to about 10% by weight of the pharmaceutical composition.

In some embodiments, the enteric coat contains other excipients, including, without limitation, one or more of the plasticizers, glidants, pH adjustment components, or surfactants described herein.

In some embodiments, the pharmacological agent coat further includes one or more of the excipients described herein. For example, in some embodiments, the pharmacological agent coat includes one or more of the binders described herein. In one embodiment, the binder is the same binder as is used in the core. In other embodiments, the binder is different from the binder used in the core. In one embodiment, the binder is Opadry II Clear.

The coatings are applied to the composition using conventional coating methods and equipment. For example, the coatings may be applied to a capsule using a coating pan, an airless spray technique, fluidized bed coating equipment, rotary granulator or the like. In some embodiments, the pharmaceutical composition is a co-compressed tablet, where the coating is compressed onto the tablet core. Detailed information concerning materials, equipment and processes for preparing coated dosage forms may be found in Pharmaceutical Dosage Forms: Tablets, Eds. Lieberman et al. (New York: Marcel Dekker, Inc., 1989), and in Ansel et al., Pharmaceutical Dosage Forms and Drug Delivery Systems, 6^{th} Edition (Media, PA: Williams & Wilkins, 1995). The coating thickness, as noted above, must be sufficient to ensure that the oral dosage form remains intact until the desired site of delivery in the lower intestinal tract is reached.

The coatings are prepared by combining the components of each coating layer. In some embodiments, the components are prepared as a dry blend, a solution, a suspension, or a dispersion. The mixture containing the components of each coating layer is then applied to the pellet or bead using the techniques described above.

For example, in one embodiment, a dispersion containing about 3% w/w to about 50% w/w, or about 5% to about 30% w/w, or about 10% w/w, or about 20% w/w, or about 15% w/w of the sustained release component is prepared to make the sustained release coat, which is then applied to the core unit and/or the pharmacological agent layer or coat.

In one embodiment, a dispersion from about about 3% w/w to about 50% w/w, or about 10% w/w to about 40% w/w, or about 10% w/w to about 15% w/w of the enteric coating polymer is prepared to make the enteric coat.

Exemplary binders include, without limitation, any coating materials that form a coating on the pharmaceutical composition and/or facilitate release of the pharmacological agent. In some embodiments, the binder includes or more of: hypromellose, polyvinylpyrrolidone, methylcellulose, hydroxypropyl cellulose, hydroxyethyl cellulose, carboxymethyl cellulose, other celluloses, starches and starch derivatives (e.g., maltodextrin), and polyvinyl alcohol. In one embodiment, the binder is hypromellose. In another embodiment, the binder is Opadry II. In some embodiments, the binder further includes one or more pharmaceutically acceptable plasticizers or glidants, including but not limited to those described herein. In one embodiment, the binder is commercially available mixture of one or more binder materials, such as Opadry II Clear. Prior to being applied to the bead, the binder can be in the form of a premix, a solution or a suspension containing one or more of the binder materials described herein. In some embodiments, the binder is present in the composition in an amount ranging from about 0.01% to about 20% by weight of the pharmaceutical composition. In some embodiments, the binder is present in the composition in an amount ranging from about 0.01% to about 15% by weight of the pharmaceutical composition. In some embodiments, the binder is present in the composition in an amount ranging from about 0.01% to about 10% by weight of the pharmaceutical composition. In some embodiments, the binder is present in the composition in an amount ranging from about 0.1% to about 5% by weight of the pharmaceutical composition. In some embodiments, the binder is present in the composition in an amount ranging from about 0.1% to about 3% by weight of the pharmaceutical composition. In some embodiments, the binder is present in the composition in an amount that is less than about 5% by weight, less than about 4% by weight, less than about 3% by weight, less than about 2% by weight or less than about 1% by weight.

In some embodiments, the pharmaceutical composition contains a plasticizer to prevent the formation of pores and cracks in the coating that would permit the penetration of the gastric fluids. Suitable plasticizers include, but are not limited to, triethyl citrate (e.g., CITROFLEX 2), dibutyl sebecate, propylene glycol (e.g., CARBOWAX 400 (polyethylene glycol 400)), triacetin (glyceryl triacetate), sorbitol, tributyl citrate, acetyltriethyl citrate (e.g., CITROFLEC A2), dibutyl phthalate, triethanolamine, diethyl phthalate, acetylated monoglycerides, glycerol, and fatty acid esters. In particular, a coating comprised of an anionic carboxylic acrylic polymer will typically contain less than about 50% by weight, or less than about 30% by weight, or from about 1% to about 25% by weight, or from about 5% to about 10% by weight, based on the total weight of the coating, of a plasticizer, particularly dibutyl phthalate, polyethylene glycol, triethyl citrate and triacetin. In one embodiment, the plasticizer is triethyl citrate.

When present, the plasticizer is typically present in the composition in an amount ranging from about 0.01% to about 20% by weight of the pharmaceutical composition. In some embodiments, the plasticizer component is present in the composition in an amount ranging from about 0.01% to about 10%, or about 0.01% to about 10% by weight. In some embodiments, the plasticizer component is present in the composition in an amount ranging from about 0.01% to about 5% or from about 0.1% to about 3%. In some embodiments, the plasticizer component is present in the composition in an amount that is less than about 1%.

Exemplary glidants include, without limitation, mono- and di-glycerides, talc, silicon dioxide, silicates, stearic acid, starch, cellulose, lactose, stearates, calcium phosphates, magnesium carbonate, magnesium oxide, and silicon dioxide aerogels. In certain embodiments, the glidant is selected from mono- and di-glycerides. When present, in some embodiments, the glidant is present in the composition in an amount ranging from about 0.01% to about 20% by weight of the pharmaceutical composition. In some embodiments, the glidant is present in the composition in an amount ranging from about 0.01% to about 10%. %. In some embodiments, the glidant is present in the composition in an amount ranging from about 0.1% to about 5%. In some embodiments, the glidant is present in the composition in an amount ranging from about 0.01% to about 3%. In some embodiments, the glidant is present in the composition in an amount that is less than about 1%.

In some embodiments, one or more of the coatings of the pharmaceutical composition also include a pH adjustment agent. The pH adjustment agent serves to adjust the pH of the enteric coating suspension such that the coating suspension has the appropriate level of neutralization to release the pharmacological agent at approximately pH 5.5. In some embodiments, the pH adjustment agent is a basic pH modifier including, without limitation, sodium hydroxide, potassium hydroxide, and ammonium hydroxide. When present, the pH adjustment component is typically present in the composition in an amount ranging from about 0.01% to about 20% by weight of the pharmaceutical composition. In some embodiments, the pH adjustment component is present in the composition in an amount ranging from about 0.01% to about 10%. In some embodiments, the pH adjustment component is present in the composition in an amount ranging from about 0.01% to about 2%. In some embodiments, the pH adjustment component is present in the composition in an amount that is less than about 1%.

Exemplary surfactants include, without limitation, ionic surfactants, nonionic surfactants or a mixture thereof. Ionic surfactants include, without limitation, sodium lauryl sulfate, dioctyl sodium sulfosuccinate and mixtures thereof. Nonionic surfactants include, without limitation, polyoxyethylene alkyl ether, polyoxyethylene alkyl ester, polysorbates (e.g., Tween 80) and mixtures thereof. Other surfactants include, without limitation, sugar esters, poloxamer, docusate sodium, polyoxyethylene stearates, sorbitan fatty acid esters, and vitamin E tocopherol polyethylene glycol succinate (TPGS). In one embodiment, the surfactant is polysorbate.

When present, in some embodiments, the surfactant is present in the composition in an amount ranging from about 0.001% to about 10% by weight of the pharmaceutical composition. In some embodiments, the surfactant component is present in the composition in an amount ranging from about 0.001% to about 5%. In some embodiments, the surfactant component is present in the composition in an amount ranging from about 0.01% to about 3%. In some embodiments, the surfactant component is present in the composition in an amount ranging from about 0.01% to about 2%. In some embodiments, the surfactant component is present in the composition in an amount that is less than about 1%.

In certain embodiments, the invention provides unit dosage forms including one or more compounds of Formula I or Formula II and a coating. In some embodiments, the coating affects the degradation of the composition to provide for release of the compound over a period of time. As used herein, “degradation” refers to degradation of the composition that results in release of at least a portion of the pharmacological agent. In some embodiments, the composition degrades in whole or in part to release the pharmacological agent. In some embodiments, the coating degrades to release the pharmacological agent. In some nonlimiting embodiments, the coating dissolves at a particular rate to provide release of the pharmacological agent over a period of time. In other nonlimiting embodiments, the composition facilitates diffusion of the pharmacological through the one or more coatings, to provide release of the agent over a period of time.

In some embodiments, the pharmaceutical compositions degrade so that the pharmacological agent is released to provide a Cₘₐₓ of the pharmacological agent between about 0.5 and about 18 hours after administration. In some embodiments, the pharmaceutical compositions degrade so that the pharmacological agent is released to provide a Cₘₐₓ of the pharmacological agent between about 2 and about 16 hours after administration. In some embodiments, the pharmaceutical compositions degrade so that the pharmacological agent is released to provide a Cₘₐₓ of the pharmacological agent between about 4 and about 12 hours after administration.

In some embodiments, the invention provides pharmaceutical compositions of compounds of Formula I or Formula II, characterized by the composition degrading such that not more than about 15% of the pharmacological agent is released in about 2 hours, determined by measuring plasma concentration. In some embodiments, not more than about 25% of the pharmacological agent is released in about 2 hours. In some embodiments, not more than about 35% of the pharmacological agent is released in about 2 hours. In some embodiments, not more than about 45% of the pharmacological agent is released in about 2 hours. In some embodiments, not more than about 50% of the pharmacological agent is released in about 2 hours. In certain embodiments, the pharmaceutical compositions degrade such that about 0.5% to about 25% of the pharmacological agent is released in about the first two hours following administration. In certain embodiments, the pharmaceutical compositions degrade such that about 25% to about 50% of the pharmacological agent is released in about the first two hours following administration.

In some embodiments, the invention provides pharmaceutical compositions of compounds of Formula I or Formula II, characterized by the composition degrading such that at least about 60% of the pharmacological agent is released in about 8 hours, determined by measuring plasma concentration. In some embodiments, at least about 75% of the pharmacological agent is released in about 8 hours. In some embodiments, at least about 80% of the pharmacological agent is released in about 8 hours. In some embodiments, at least about 85% of the pharmacological agent is released in about 8 hours.

In some embodiments, the invention provides pharmaceutical compositions of compounds of Formula I or Formula II, characterized by a dissolution profile such that not more than about 15% of the pharmacological agent is dissolved in about 2 hours. In some embodiments, not more than about 25% of the pharmacological agent is released in about 2 hours. In some embodiments, not more than about 35% of the pharmacological agent is dissolved in about 2 hours. In some embodiments, not more than about 45% of the pharmacological agent is dissolved in about 2 hours. In some embodiments, not more than about 50% of the pharmacological agent is dissolved in about 2 hours. In some embodiments, from about 15% to about 50%, or about 15% to about 40%, or about 15% to about 30%, or about 25% of the pharmacological agent is dissolved in about 2 hours.

In some embodiments, the invention provides pharmaceutical compositions of compounds of Formula I or Formula II, characterized by a dissolution profile such that at least about 60% of the pharmacological agent is dissolved in about 8 hours. In some embodiments, at least about 75% of the pharmacological agent is dissolved in about 8 hours. In some embodiments, at least about 80% of the pharmacological agent is dissolved in about 8 hours. In some embodiments, at least about 85% of the pharmacological agent is dissolved in about 8 hours. In some embodiments, from about 60% to about 85%, or about 70% to about 80%, or about 75% of the pharmacological agent is dissolved in about 8 hours.

In some embodiments, dissolution is determined using simulated gastrointestinal media, using for example United States Pharmacopoeia (“USP”) Apparatus 2 at 75 rpm. In some embodiments, the pharmaceutical compositions are subjected to pH˜1 (0.01N HCl) for approximately two hours, thus simulating the low pH environment of the stomach. This step is followed by exposure to a buffer of pH˜6.8 and about 1% sodium lauryl sulfate for approximately six more hours, which simulates the higher pH of the small intestine. In some embodiments, the second buffer is at pH˜6.0 and about 2% sodium lauryl sulfate for about 10 hours. In some embodiments, the pharmaceutical compositions employ a single-stage dissolution at pH˜4.5.

In some embodiments, the compositions provide release of a majority of the pharmacological agent at a pH of greater than about 6, as demonstrated by in vitro dissolution studies.

In certain embodiments, the present invention provides a multiple particulate (or multiparticulate) formulation comprising a plurality of beads described herein above. Without being bound to a particular theory, it is thought that the use of multiple particulate formulation provides a dispersion of individual beads within the stomach, resulting in the introduction of the enteric coated particles into the small intestine over a period of time, and thus achieving a desirable sustained-release profile.

In some embodiments, the multiple particulate formulation is achieved by mixing same or different types of beads in one dosage form. For example, one group of beads may contain the pharmacological agent, a sustained release coat, and an enteric coat while another group of beads contains a layer of pharmacological agent only. When mixed together in the appropriate amount in a single unit dosage form, a release profile similar to that observed for a dosage form containing one type of bead (i.e., four-layer bead) is obtained.

In certain embodiments, the multiple particulate formulation comprises:

(A) at least one first bead comprising:

(i) a core unit of a substantially water-soluble or water-swellable inert material;

(ii) a first layer on the core unit comprising a pharmacological agent, an acidifier, and an optional binder;

(iii) a second layer of sustained-release coat covering the first layer; and

(iv) a third layer of enteric coat on the second layer; and

(B) at least one second bead comprising the pharmacological agent optionally covered by a sustained-release coat.

In certain other embodiments, the multiple particulate formulation comprises:

(A) at least one first bead comprising:

(i) a core unit comprising a mixture of a substantially water-soluble or water-swellable inert material, a pharmacological agent, an acidifier and an optional binder;

(ii) a first layer of sustained-release coat on the core unit; and

(iii) a second layer of enteric coat covering the first layer; and

(B) at least one second bead comprising the pharmacological agent optionally covered by a sustained-release coat.

In some embodiments, the ratio between the pharmacological agent contained in the second bead to that contained in the first bead is from about 15% to about 40% w/w. In some embodiments, the ratio is from about 20% to about 35% w/w. In some other embodiments, the ratio is from about 20% to about 35% w/w. In yet other embodiments, the ratio is from about 25% to about 30% w/w.

In some embodiments, the multiple particulate formulation has a dissolution profile characterized in that about 20% to about 45% by weight of the pharmacological agent is released after about 2 hours and more than about 60% by weight of the pharmacological agent is released after about 8 hours, in simulated gastrointestinal media, such as Apparatus 2 described in the United States Pharmacopoeia (USP29-NF24, page 2673) having a rotation speed of 75 rpm, and a two stage dissolution medium containing (i) pH˜1 buffer for about two hours, and followed by (ii) pH˜6.8 buffer with 1% SLS (sodium lauryl sulfate) for about 6 additional hours.

In certain embodiments, the multiple particulate formulation comprises:

a) the water-soluble or water-swellable inert material comprises from about 60% to about 90% by weight of the total formulation;

b) the pharmacological agent comprises from about 1% to about 25% by weight of the total formulation;

c) the acidifier comprises from about 0.5% to about 10% by weight of the total formulation d;

d) the sustained-release coat comprises from about 1% to about 20% by weight of the total formulation;

e) the binder comprises from about 0.1% to about 5% by weight of the total formulation; and

f) the enteric coat comprises from about 0.5% to about 20% by weight of the total formulation, in which the enteric coat contains from about 0.5% to about 15% of an enteric coating polymer or copolymer by weight of the bead, from about 0.01% to about 2% of a pH adjustment agent by weight of the bead, from about 0.1% to about 5% of a glidant by weight of the bead, from about 0.1% to about 3% of a plasticizer by weight of the bead, and from about 0.01% to about 2% of a surfactant by weight of the bead.

In certain other embodiments, the pharmacological agent comprises from about 1% to about 10% by weight of the total formulation; the acidifier comprises from about 1% to about 5% by weight of the total formulation; the sustained-release coat comprises from about 5% to about 15% by weight of the total formulation; and the enteric coat comprises from about 1% to about 15% by weight of the total formulation.

In some embodiments, the pharmaceutical compositions include single dosage forms. In other embodiments, the pharmaceutical compositions include multiple dosage forms. In some embodiments, the pharmaceutical compositions include multiple particulate formulations (or multiparticulates) comprising a plurality of beads. The beads can be same or different. The beads can be encapsulated or compressed into a final form.

The pharmaceutical dosage forms described herein may be in any suitable solid dosage form for oral administration. Nonlimiting examples of suitable solid dosage forms include powders, capsules, tablets, pills, troches, cachets, and pellets. In some embodiments, the pharmaceutical composition is a capsule or a tablet. In some embodiments, the composition is coated, for example with an enteric coating and/or a sustained release coating. In some embodiments, the composition is a multilayer dosage form. Some embodiments comprise a bi-layer dosage form. In other embodiments, the composition comprises a tri-layer dosage form. In some embodiments, the composition is prepared for sustained release (i.e., a sustained release composition).

The capsule material may be either hard or soft, and as will be appreciated by those skilled in the art, typically includes a tasteless, easily administered and water soluble compound, such as gelatin, starch polysaccharide or a cellulosic material. In some embodiments, the capsules are sealed, such as with gelatin bands or the like. See, for example, Remington: The Science and Practice of Pharmacy, 20^{th} Edition (Easton, Pa.: Mack Publishing Company, 2000), which describes materials and methods for preparing encapsulated pharmaceuticals. The solid dosage forms described herein also optionally include one or more excipients or additives for forming a solid dosage form of said pharmaceutical composition. Suitable optional additives include binders, plasticizers, glidants, pH adjustment components, surfactants, detackifiers, antifoaming agents, lubricants (e.g., magnesium stearate), and stabilizers (e.g., hydroxypropylcellulose, acids and bases) to solubilize or disperse the coating material, and to improve coating performance and the coated product, or mixtures thereof.

In one nonlimiting embodiment, a coated pharmaceutical composition includes a capsule containing the pharmacological agent. The capsule is coated with an enteric coat that includes an enteric coat component and/or a sustained release coat that includes a sustained release component. In some embodiments, an acidifier is located in the capsule with the pharmacological agent. In other embodiments, the acidifier is located in the coating. In some embodiments, the acidifier is located in the enteric coat. In other embodiments, the acidifier is located both inside the capsule and in the coating.

In another nonlimiting embodiment, a coated pharmaceutical composition includes a coated tablet or mini-tablet. The core of the tablet contains the pharmacological agent, with a sustained release coat and/or an enteric coat added to the exterior of the tablet. The sustained release coat includes the sustained release component, while the enteric coat contains the enteric coat component. In some embodiments, the acidifier is located in the core of the tablet with the pharmacological agent. In other embodiments, the acidifier is located in the coating. In other embodiments, the acidifier is located in the enteric coat. In further embodiments, the acidifier is located both in the core of the tablet and in the coating.

In some embodiments, the pharmaceutical composition includes a plurality of coated pellets (e.g., particulates, particles, beads, or spheroids). Each bead includes a pharmaceutically effective amount of a pharmacological agent and an acidifier. In some embodiments, each bead also includes a sustained release coat and/or an enteric coat. The sustained release coat includes the sustained release component, while the enteric coat contains the enteric coat component. In some embodiments, the acidifier is located in the core of the bead with the pharmacological agent. In other embodiments, the acidifier is located in the coating. In other embodiments, the acidifier is located in the enteric coat. In further embodiments, the acidifier is located both in the core of the bead and in the coating.

In some embodiments, the beads are incorporated into a capsule to achieve a predetermined dose of the pharmacological agent. The capsules are composed of any pharmaceutically acceptable carrier material known to those of skill in the art. Exemplary capsule materials include, without limitation, hypromellose, gelatin, pullulan, or starch. In other embodiments, the beads are incorporated in a pouch or similar package. In some embodiments, the beads are compressed into a tablet by blending with one or more plastically deforming excipients, such as cellulose or starch that facilitate compression and cushion the beads without disturbing the coating on the surface of the bead. In some embodiments, when the beads are compressed into a tablet, the beads are coated with a binder coating as described herein. The binder coating further protects the integrity of the beads during the compression process.

In some embodiments, the coated pharmaceutical compositions described herein also include a pharmacological agent coat. The pharmacological agent coat includes a portion of the total dosage of the pharmacological agent of the composition. In some embodiments, the pharmacological agent coat forms the outermost layer of the pharmaceutical composition.

In some embodiments, the coated pharmaceutical compositions described herein also include a binder coating. The binder coating includes a fast dissolving, water soluble coating component. The binder coating includes any of the binder materials described herein. In some embodiments, the binder coating includes an Opadry material. In some embodiments, the binder coating forms the outermost layer of the pharmaceutical composition.

The amount of pharmacological agent in the oral unit dosage form, with as a single or multiple dosage, is an amount that is effective for treating or preventing a 5-HT_{1A}-related disorder. As one of skill in the art will recognize, the precise dose to be employed will depend on a variety of factors, examples of which include the condition itself, the seriousness of the condition being treated, the particular composition used, as well as various physical factors related to the individual being treated. In vitro or in vivo assays can optionally be employed to help identify optimal dosage ranges. To determine the effective amount of the composition being administered, a physician may, for example, evaluate the effects of a given composition of a compound of Formula I or Formula II in the patient by incrementally increasing the dosage until the desired symptomatic relief level is achieved. The dose regimen may then be further modified to achieve the desired result. For oral administration, in some embodiments, the compositions described herein are incrementally increased in a patient in an amount from about 0.001 mg/kg to about 10 mg/kg until the desired symptomatic relief level is achieved. In some embodiments, the patient is administered the compositions described herein as a single oral dose (e.g., one 10 mg tablet or capsule) or as a multiple oral dose (e.g., three 3 mg tablets or capsules; two 5 mg tablets or capsules; four 2.5 mg tablets or capsules).

In some embodiments, however, a dosage (whether in unit or multiple dosage form) for daily oral administration will range from about range from about 0.001 mg to about 600 mg per day, in one embodiment, from about 1 mg to about 600 mg per day, in another embodiment, from about 10 mg to about 400 mg per day, in another embodiment, from about 10 mg to about 200 mg per day, in another embodiment, from about 10 mg to about 100 mg per day, in another embodiment, from about 1 mg to about 100 mg per day, in another embodiment, from about 1 mg to about 50 mg per day, in another embodiment, from about 1 mg to about 25 mg per day, and in another embodiment, from about 1 mg to about 10 mg per day. In one embodiment, the dosage for daily oral administration will be 2.5 mg per day, 5 mg per day, 7.5 mg per day, 10 mg per day, 15 mg per day, 20 mg per day, 25 mg per day, 30 mg per day, 35 mg per day, 40 mg per day, 45 mg per day, or 50 mg per day.

The oral unit dosage forms described herein (tablets or capsules) generally contain from about 0.25 mg to about 500 mg of the pharmacological agent. In some embodiments, the oral unit dosage forms contain from about 0.25 mg to about 400 mg of the pharmacological agent, or about 0.25 mg to about 300 mg of the pharmacological agent, or about 0.25 mg to about 250 mg of the pharmacological agent, or about 0.25 mg to about 200 mg of the pharmacological agent, or about 0.25 mg to about 100 mg of the pharmacological agent, or about 0.25 mg to about 75 mg of the pharmacological agent, or about 0.25 mg to about 50 mg of the pharmacological agent, or about 0.25 mg to about 25 mg of the pharmacological agent, or about 0.25 mg to about 15 mg of the pharmacological agent. In some embodiments, the oral unit dosage forms contain about 0.25 mg of the pharmacological agent, or about 0.5 mg of the pharmacological agent, or about 0.75 mg of the pharmacological agent, or about 1 mg of the pharmacological agent, or about 1.5 mg of the pharmacological agent, or about 2 mg of the pharmacological agent, or about 2.5 mg of the pharmacological agent, or about 3 mg of the pharmacological agent, or about 3.5 mg of the pharmacological agent, or about 4 mg of the pharmacological agent, or about 4.5 mg of the pharmacological agent, or about 5 mg of the pharmacological agent, or about 6 mg of the pharmacological agent, or about 7 mg of the pharmacological agent, or about 8 mg of the pharmacological agent, or about 9 mg of the pharmacological agent, or about 10 mg of the pharmacological agent, or about 15 mg of the pharmacological agent, or about 20 mg of the pharmacological agent, or about 25 mg of the pharmacological agent, or about 50 mg of the pharmacological agent, or about 100 mg of the pharmacological agent.

In some embodiments, the compound of Formula I or Formula II is present in the solid pharmaceutical dosage form at a level of about 1% by weight to about 75% by weight based on the total weight of the pharmaceutical composition. In some embodiments, the compound is present at a level of about 1% by weight to about 50% by weight, about 1% by weight to about 25% by weight, about 1% by weight to about 15% by weight, or at a level of about 1% by weight to about 10% by weight, based on the total weight of the solid pharmaceutical dosage form.

In some embodiments, the pharmaceutical compositions described herein further include one or more other pharmaceutical agents. In some embodiments, the other therapeutic agent is administered in an effective amount. In some embodiments, the one or more other pharmaceutical agents are separate from the pharmaceutical compositions described herein. In some embodiments, the one or more other pharmaceutical agents are administered simultaneously and/or successively with the pharmaceutical compositions described herein.

Effective amounts of the other therapeutic agents are known to those skilled in the art. However, it is within the skilled artisan's purview to determine the other therapeutic agent's optimal effective amount range. The compound or a pharmaceutically acceptable salt of the compound and the other therapeutic agent can act additively or, in some embodiments, synergistically. In one embodiment, where another therapeutic agent is administered with the compound of Formula I, the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically. In some cases, the patient in need of treatment is being treated with one or more other therapeutic agents. In some cases, the patient in need of treatment is being treated with at least two other therapeutic agents.

In one embodiment, the other therapeutic agent is selected from the group consisting of one or more anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677, the disclosures of which are incorporated by reference in their entirety. Suitable atypical anti-depressants include bupropion, lithium, nefazodone, trazodone and viloxazine. Suitable NK-1 receptor antagonists include those referred to in International Patent Publication WO 01/77100, which is incorporated by reference in its entirety.

Anti-anxiety agents that can be used in combination with the active compounds of this invention include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that are used in combination with the active compounds of this invention include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenytoin, primidone, or carbamazepine.

Cognitive enhancers that are co-administered with the pharmaceutical compositions described herein include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5-HT receptor modulators, including other 5-HT_{1A} antagonist compounds and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) inhibitors (e.g., PDE IV inhibitors, roflumilast, arofylline, cilomilast, rolipram, RO-20-1724, theophylline, denbufylline, ARIFLO, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In some embodiments, the pharmaceutical compositions described herein are included in kits or packages of pharmaceutical formulations designed for use in the regimens and methods described herein. In some embodiments, these kits are designed for daily oral administration over the specified term or cycle of administration, in some embodiments for the number of prescribed oral administrations per day, and organized so as to indicate a single oral formulation or combination of oral formulations to be taken on each day of the regimen or cycle. In some embodiments, each kit will include oral tablets or capsules to be taken on each of the days specified, in some embodiments one oral tablet or capsule will contain each of the combined daily dosages indicated and in other embodiments, the administrations of the separate compounds will be present in separate formulations or compositions. In some embodiments, shall have a calendar or days-of-the-week designation directing the administration of the appropriate compositions on the appropriate day or time.

In one embodiment, the pharmaceutical compositions described herein are useful as 5-HT_{1A} receptor antagonists. In another embodiment, the pharmaceutical compositions are useful as 5-HT_{1A} receptor agonists. Compounds that are 5-HT_{1A} antagonists and/or agonists can readily be identified by those skilled in the art using numerous art-recognized methods, including standard pharmacological test procedures such as those described herein. Accordingly, the pharmaceutical compositions described herein are useful for treating a mammal with a 5-HT_{1A}-related disorder. One non-limiting example of a disorder that 5-HT_{1A} receptor antagonists are useful for treating is cognition-related disorder, while a non-limiting example of a disorder that 5-HT_{1A} receptor agonists are useful for treating is anxiety-related disorder. In some embodiments, the pharmaceutical compositions described herein are useful for improving cognitive function or cognitive deficits. Examples of improvements in cognitive function include, without limitation, memory improvement and retention of learned information. Accordingly, the pharmaceutical compositions described herein are useful for slowing the loss of memory and cognition and for maintaining independent function for patients afflicted with a cognition-related disorder. Thus, in one embodiment, the pharmaceutical compositions described herein that contain compounds that act as 5-HT_{1A} receptor antagonists are useful for treating a mammal with a cognition-related disorder. In one embodiment, the pharmaceutical compositions that contain compounds that act as 5-HT_{1A} receptor antagonists are useful for improving the cognitive function of a mammal. Similarly, in one embodiment, pharmaceutical compositions that include compounds that act as 5-HT_{1A} receptor agonists are useful for treating a mammal with an anxiety-related disorder.

In one embodiment, the invention provides a method for treating a 5-HT_{1A}-related disorder, including administering to a mammal in need thereof a pharmaceutical composition described herein in an amount effective to treat a 5-HT_{1A}-related disorder. In one embodiment, the invention provides a method for treating a cognition-related disorder, including administering to a mammal in need thereof a pharmaceutical composition described herein in an amount effective to treat a 5-HT_{1A}-related disorder. In one embodiment, the invention provides a method for treating an anxiety-related disorder, including administering to a mammal in need thereof a pharmaceutical composition described herein in an amount effective to treat a 5-HT_{1A}-related disorder.

In one embodiment, the invention provides a method for treating Alzheimer's disease, including administering to a mammal in need thereof a pharmaceutical composition described herein in an amount effective to treat Alzheimer's disease. In one embodiment, the method for treating Alzheimer's disease includes administering a second therapeutic agent. In some embodiments, the second therapeutic agent is an anti-depressant agent, an anti-anxiety agent, an anti-psychotic agent, or a cognitive enhancer.

In one embodiment, the invention provides a method for treating mild cognitive impairment (MCI), includes administering to a mammal in need thereof a compound or a pharmaceutical composition described herein in an amount effective to treat mild cognitive impairment (MCI). In one embodiment, the method for treating MCI includes administering a second therapeutic agent. In some embodiments, the second therapeutic agent is an anti-depressant agent, an anti-anxiety agent, an anti-psychotic agent, or a cognitive enhancer.

In one embodiment, the invention provides a method for treating depression, including administering to a mammal in need thereof a pharmaceutical composition described herein in an amount effective to treat depression. In one embodiment, the method for treating depression includes administering a second therapeutic agent. In some embodiments, the second therapeutic agent is an anti-depressant agent, an anti-anxiety agent, an anti-psychotic agent, or a cognitive enhancer.

In one embodiment, the a pharmaceutical composition described herein are useful for treating sexual dysfunction, e.g., sexual dysfunction associated with drug treatment such as drug treatment with an antidepressant, an antipsychotic, or an anticonvulsant. Accordingly, in one embodiment, the invention provides a method for treating sexual dysfunction associated with drug treatment in a patient in need thereof. The method includes administering to the patient an effective amount of one or more of the pharmaceutical compositions disclosed herein. In some embodiments, the drug treatment is antidepressant drug treatment, antipsychotic drug treatment, or anticonvulsant drug treatment.

In certain embodiments, the drug associated with sexual dysfunction is a selective serotonin reuptake inhibitor (SSRI) (for example, fluoxetine, citalopram, escitalopram oxalate, fluvoxamine maleate, paroxetine, or sertraline), a tricyclic antidepressant (for example, desipramine, amitriptyline, amoxipine, clomipramine, doxepin, imipramine, nortriptyline, protriptyline, trimipramine, dothiepin, butriptyline, iprindole, or lofepramine), an aminoketone class compound (for example, bupropion). In some embodiments, the drug is a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT_{2A} receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenytoin, primidone, or carbamazepine. In some cases, the patient in need of treatment for sexual dysfunction is being treated with at least two drugs that are antidepressant drugs, antipsychotic drugs, anticonvulsant drugs, or a combination thereof.

In some embodiments of the invention, the sexual dysfunction comprises a deficiency in penile erection.

The invention also provides a method of improving sexual function in a patient in need thereof. The method includes administering to the patient a pharmaceutically effective amount of a pharmaceutical composition described herein.

The pharmaceutical compositions described herein are also useful in the manufacture of medicaments for treating a 5-HT_{1A}-related disorder in a mammal. Similarly, the pharmaceutical compositions described herein are also useful in the manufacture of medicaments for treating a cognition-related disorder in a mammal. Also, the pharmaceutical compositions described herein are also useful in the manufacture of medicaments for treating an anxiety-related disorder in a mammal.

The following, specific, non-limiting examples are provided to further illustrate the embodiments described herein. The reagents and intermediates used herein are either commercially available or prepared according to standard literature procedures. The materials, methods, and examples presented herein are illustrative and are not intended to limit the scope of the invention. All publications, patent applications, patents, and other references mentioned herein are incorporated by reference in their entireties.

### EXAMPLES

### Example 1

### Preparation of 4-Layer Controlled-Release Beads

A single process operation was used to prepare four-layer controlled-release beads (or “pellets”). A schematic depiction of the 4-layer beads is provided in FIG. 1. The most inner layer contained about 70% to about 75% of the compound of interest in the composition. Citric acid was added to the inner layer containing the compound, to facilitate solubility. A second layer was applied on top of the citric acid and the compound, containing Surelease to sustain release of the compound at higher pH in the lower gastrointestinal tract. A third layer was then added, containing Eudragit, an enteric coating added to prevent release in the stomach. The final, outermost layer contained about 20% to about 25% of the compound, intended to be released in the stomach shortly after administration.

The process proceeded as follows. In the first step, sugar spheres were loaded into a fluid bed processor with a Wurster insert. In the second step, a suspension of 9.3% w/w 5-fluoro-8-{4-[4-(6-methoxyquinolin-8-yl)piperazin-1-yl]piperidin-1-yl}quinoline trisuccinate in Opadry II Clear with citric acid was added to the sugar spheres. The suspension was prepared by dissolving citric acid anhydrous (100 g, 9.3% by weight of the suspension) in water. Then, 5-fluoro-8-{4-[4-(6-methoxyquinolin-8-yl)piperazin-1-yl]piperidin-1-yl}quinoline trisuccinate (175.13 g, 16.29% by weight of the suspension (salt), equivalent to 100 g, 9.3% of the suspension (free base)) was suspended in the solution and stirred until homogenous. To this mixture, an 11.11% w/w aqueous solution of Opadry II Clear was added (400 g, 37.2% by weight of the suspension).

In step 3, a 15% w/w suspension of Surelease sustained-release coating was applied to the coated spheres from step 2. The Surelease coating suspension was prepared by adding 600 g of Surelease E-7-190010, Global (25% w/w solid contents) in 400 g purified water. In-process dissolution was performed to determine the adequacy of the applied coating. Additional Surelease coating was applied as necessary.

In step 4, an enteric coating suspension was applied to the coated spheres from step 3. The enteric coating suspension contained 13.13% w/w Eudragit L30D-55. It was prepared by mixing Eudragit L30D-55 dispersion (44.44 g), containing 30% w/w solids, with triethyl citrate (3.33 g), 10% Imwitor 90K dispersion (133.33 g), 1N sodium hydroxide (22.0 g), and purified water (1000 g). The mixture was prepared by placing the Eudragit dispersion in a vessel equipped with a low shear (Lightnin type) or suitable mixer. The Eudragit dispersion was screened through a hand screen prior to dispensing to remove any agglomerates that may clog the spray nozzle. The triethyl citrate and Imwitor 900K dispersion was gradually added under moderate agitation, followed by an addition of 300 g purified water, with continued agitation. Sodium hydroxide was added gradually to adjust the pH of the suspension to approximately 5.3. No more than about 22 g of IN sodium hydroxide per 100 g of enteric coating is added in this step. The entire suspension was then brought to the total theoretical weight with purified water and mixed to achieve a uniform mixture.

The percentage of mono- and di-glycerides, triethyl citrate, polysorbate 80, and 1N NaOH in the final coated composition (dried) was as follows. Values presented are weight percentages relative to the total weight of Eudragit in the final composition. Imwitor 900K mono- and di-glycerides was 10.0% w/w (Eudragit). Triethyl citrate was 7.5% w/w (Eudragit), taking into account the contribution from the Imwitor mono- and di-glycerides, which contain approximately 0.33% w/w triethyl citrate. Polysorbate 80 was 1.0% w/w (Eudragit). 1N NaOH was 0.66% w/w (Eudragit).

In-process dissolution was performed to determine the adequacy of the applied enteric coating. Additional enteric coating was applied as necessary. The in-process dissolution can also be performed prior to the application of the enteric coating suspension.

In step 5, a suspension of 5.13% w/w 5-fluoro-8-{4-[4-(6-methoxyquinolin-8-yl)piperazin-1-yl]piperidin-1-yl}quinoline in Opadry II Clear was applied to the spheres of step 4. The suspension was prepared by adding 175.13 g of the trisuccinate salt of the compound (8.98% w/w, equivalent to 100 g (5.13% w/w) of the free base) in water (887.56 g, 45.51% w/w) to form a homogenous suspension, then adding 887.56 g of the 11.11% w/w Opadry II Clear solution (45.51% w/w, equivalent to 5.06% solids) described in the first step.

Following coating, the beads were encapsulated in #0E hypromellose capsule shells to the target fill weights required to arrived at 0.5 mg, 2.5 mg, and 10 mg of the compound of interest.

Table 1 provides the unit dosage details of the formulation, for each of 0.5 mg, 2.5 mg, and 10 mg capsules, adjusted based on the actual assay of the beads to achieve the proper dose.

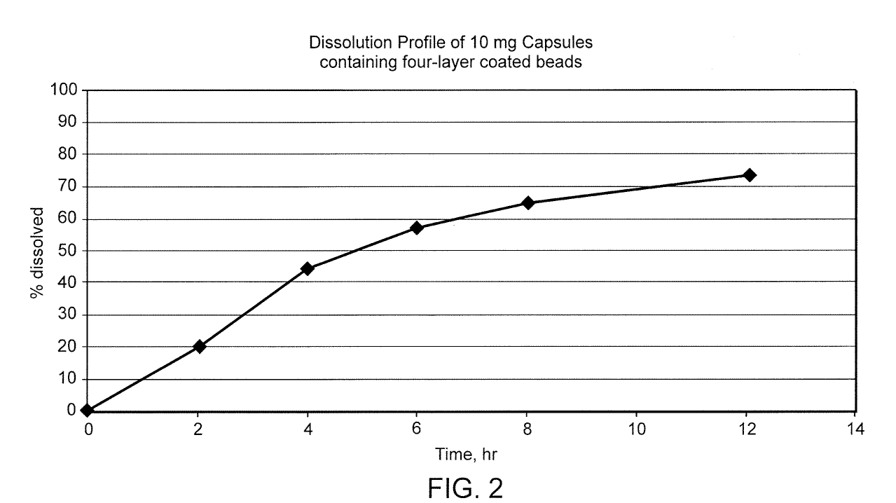

The dissolution profile of 10 mg capsules prepared according to the procedure described above was determined as follows. United States Pharmacopoeia (“USP”) Apparatus 2 at 75 rpm was used for the dissolution testing. Capsules were subjected to pH˜1 (0.01N HCl) for approximately two hours, followed by exposure to a buffer of pH˜6.0 containing about 2% sodium lauryl sulfate for approximately ten more hours. The results of the dissolution study are presented in FIG. 2 and demonstrate gradual dissolution over the course of the study, including accounting for the pH change.

TABLE 1
Composition 0.5 mg, 2.5 mg and 10 mg Capsules Including Four-Layer Coated Beads

| | | | 0.5 mg Capsules | 2.5 mg Capsules | 10 mg Capsules |
|---|---|---|---|---|---|
| | | % | Formulation | Formulation | Formulation |
| Ingredient | Function | w/w | Unit Dose (mg/capsule) | | |
| Coated Beads | | | | | |
| Sugar sphere, NF | Carrier | 78.29 | 15.72 | 78.62 | 314.48 |
| 5-fluoro-8-{4-[4-(6-methoxyquinolin-8- | Active | 3.43^{a} | 0.69^{a} | 3.44^{a} | 13.78^{a} |
| yl)piperazin-1-yl]piperidin-1-yl}quinoline | | | | | |
| trisuccinate | | | | | |
| Citric acid anhydrous | acidifier | 1.96 | 0.39 | 1.97 | 7.87 |
| Opadry II Clear Y-19-7483 | Binder | 0.87 | 0.17 | 0.87 | 3.49 |
| Purified water,^{c} | Formulation solvent | — | QS | QS | QS |
| Sustained-release Coat | | | | | |
| Surelease-E-7-19010, Global | Sustained-release coating | 8.46 | 1.70 | 8.50 | 33.98 |
| | polymer | | | | |
| Purified Water,^{c} | Formulation solvent | QS | QS | QS | QS |
| Enteric Coat | | | | | |
| Methacrylic Acid Copolymer Dispersion | Enteric coating polymer | 4.65 | (3.11 dispersion) | (15.57 dispersion) | (62.26 dispersion) |
| (Eudragit L30D-55) (30% w/w dispersion) | | | 0.93 (polymer) | 4.67 (polymer) | 18.68 (polymer) |
| Mono- and Di-Glycerides (Imwitor 900K) | Glidant | 0.46 | 0.09 | 0.46 | 1.85 |
| Triethyl Citrate | Plasticizer | 0.35 | 0.07 | 0.35 | 1.41 |
| Sodium Hydroxide | pH adjuster | 0.03 | 0.006 (solid) | 0.03 (solid) | 0.12 (solid) |
| Polysorbate 80 (Vegetable Sourced) | Surfactant | 0.05 | 0.01 | 0.05 | 0.20 |
| Purified Water,^{c} | Formulation solvent | QS | QS | QS | QS |
| Pharmacological Agent Coat | | | | | |
| 5-fluoro-8-{4-[4-(6-methoxyquinolin-8- | Active | 0.93 | 0.19 | 0.93 | 3.74 |
| yl)piperazin-1-yl]piperidin-1-yl}quinoline | | | | | |
| trisuccinate | | | | | |
| Opadry II Clear Y-19-7483 | Binder | 0.52 | 0.10 | 0.52 | 2.09 |
| Purified water,^{c} | Formulation solvent | QS | QS | QS | QS |
| Encapsulation | | | | | |
| #0E, Brown HPMC capsules | Capsule shell | | 1 capsule | 1 capsule | 1 capsule |
| | Total | 100.0 | 20.07^{b} | 100.41^{b} | 401.69^{b} |

^{a}As the free base. 3.44% of the trisuccinate salt is equivalent to 2.5% of the free base.
^{b}Fill weight.
^{c}Used in the formulation but does not appear in the finished product.

### Example 2

### Preparation of 3-Layer Coated Beads

A formulation of 3-layer coated beads was prepared in accordance with the methods described in Example 1. The 3-layer beads are similar to the 4-layer beads of Example 1, although they do not include the outermost layer of active ingredient.

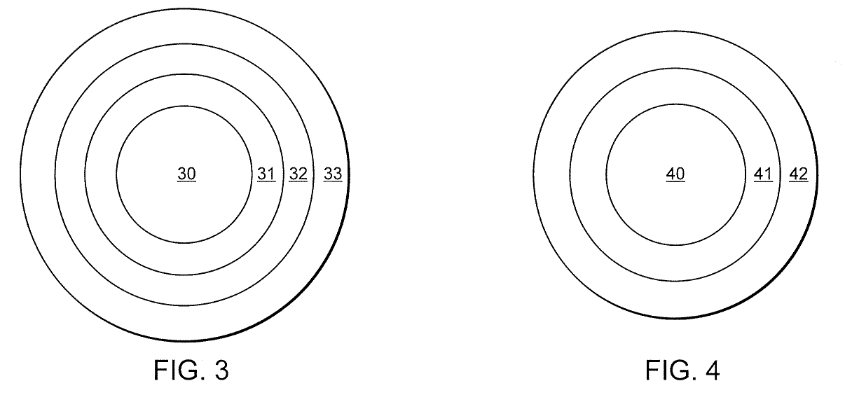

The formulation for the 3-layer beads is provided in Table 2. A schematic description of the 3-layer beads is provided in FIG. 3. FIG. 3 depicts a cross-section of a coated bead as described herein. The sugar sphere 30 is at the center of the bead, coated with a layer of the active component and citric acid 31, followed by a layer of 10.5% Surelease 32 (sustained release coating). The outermost layer 33 includes a coating of 10% Eudragit enteric coat component.

Following coating, a sufficient quantity of coated beads was encapsulated in hypromellose capsule shells to arrive at a final unit dosage of approximately 10 mg.

TABLE 2
Composition of Three Layer Coated Beads

| Ingredient | w/w % |
|---|---|
| Sugar sphere | 72.38 |
| 5-fluoro-8-{4-[4-(6-methoxyquinolin-8- | 3.24^{d} |
| yl)piperazin-1-yl]piperidin-1-yl}quinoline | |
| trisuccinate^{a} | |
| Opadry Clear II | 0.60 |
| Citric Acid | 3.67 |
| Surelease | 8.49 |
| Methacrylic Acid Copolymer Dispersion | 8.94 |
| (Eudragit L30D-55) (solid) | |
| Triethyl Citrate | 0.67 |
| Mono- and Di-Glycerides (Imwitor 900K) | 0.90 |
| Polysorbate 80 (Tween 80) | 0.09 |
| NaOH (solid) | 0.04 |
| Water^{e} | q.s. |

^{d}Equivalent to 1.83%, based on ~56.6 use at value.
^{e}Not present in final product

### Example 3

### Preparation of Sustained Release Coated Beads

A formulation of coated beads containing only an active layer and a sustained release layer was prepared in accordance with the methods described in Example 1. The sustained release coated beads are similar to the 4-layer beads of Example 1, although they do not include the outermost layer of active ingredient or the enteric coating layer.

The formulation for the 2-layer beads is provided in Table 3. A schematic description of the 2-layer beads is provided in FIG. 4. FIG. 4 depicts a cross-section of a coated bead as described herein. The sugar sphere 40 is at the center of the bead, coated with a layer of the active component and citric acid 41. The outermost layer 42, includes a coating of 4.5% Surelease (sustained release coating).

Following coating, a sufficient quantity of coated beads was encapsulated in hypromellose capsule shells to arrive at a final unit dosage of approximately 2.5 mg.

TABLE 3
Composition of Sustained Release Coated Beads

| Ingredient | w/w % |
|---|---|
| Sugar sphere | 86.840 |
| 5-fluoro-8-{4-[4-(6-methoxyquinolin-8- | 3.840^{d} |
| yl)piperazin-1-yl]piperidin-1-yl}quinoline | |
| trisuccinate^{a} | |
| Opadry Clear II | 0.680 |
| Citric Acid | 4.340 |
| Surelease | 4.310 |
| Water^{e} | q.s. |

^{d}Equivalent to 1.83%, based on ~56.6 use at value.
^{e}Not present in final product

### Example 4

### Dissolution Studies

Studies were performed to determine the dissolution profiles of the pharmaceutical compositions described herein. In one study, separate dissolution tests were performed on 10 mg capsules. In one group, four-layer capsules made according to the procedure described in Example 1 were tested. Table 4 provides the final composition of the capsules.

TABLE 4
Composition of Four-Layer Coated Beads

| Ingredient | w/w % |
|---|---|
| Sugar sphere | 73.07 |
| 5-fluoro-8-{4-[4-(6-methoxyquinolin-8- | 5.38^{d} |
| yl)piperazin-1-yl]piperidin-1-yl}quinoline | |
| trisuccinate^{a} | |
| Opadry Clear II | 1.49 |
| Citric Acid | 1.83 |
| Surelease | 8.83 |
| Eudragit L30D-55 (solid) | 7.89 |
| Triethyl citrate | 0.59 |
| Tween 80 | 0.08 |
| NaOH (solid) | 0.05 |
| Water^{e} | q.s. |

^{d}Equivalent to 3.04%, based on ~56.6% use at value.
^{e}Not present in final product

In another group, a mixture containing 50% of particles made according to Example 2 and 50% of particles made according to Example 3 was prepared.

The dissolution profiles were determined using United States Pharmacopoeia (“USP”) Apparatus 2 at 75 rpm. Each group was subjected to pH˜1 (0.01N HCl) for approximately two hours, followed by exposure to a buffer of pH˜6.8 containing about 1% sodium lauryl sulfate for approximately six more hours.

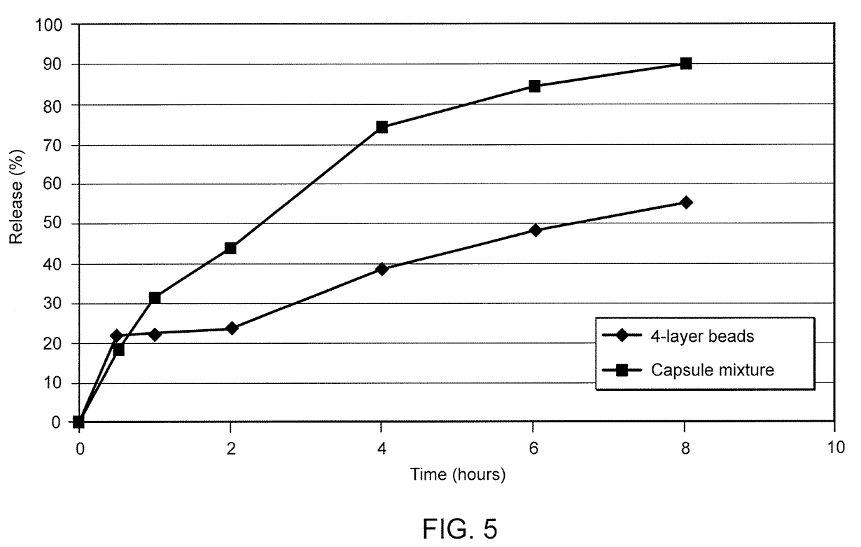

A graph of the dissolution profile of each group is provided in FIG. 5. As the data demonstrate, both groups of capsule formulations provided dissolution of the pharmacological agent over the 8 hours of the test. Dissolution ranged from about 20% to about 45% after about two hours and steadily increased cover the remainder of the study. At eight hours, between about 55% and about 90% of the pharmacological agent was dissolved.

### Example 5

### Solubility Study

The solubility of one of the compounds described herein, 5-fluoro-8-{4-[4-(6-methoxyquinolin-8-yl)piperazin-1-yl]piperidin-1-yl}quinoline, as a function of pH was determined experimentally. In the study, suspensions of about 0.5-40 mg/mL of the compound were prepared in water and various concentrations of hydrochloric acid and sodium hydroxide. The suspensions were measured for pH after equilibration at ambient temperature. The suspensions were then filtered through a suitable filter for concentrated samples and centrifuged for lower concentrations, with confirmation that the supernatants were particulate-free by laser. The filtrates were then analyzed by HPLC.

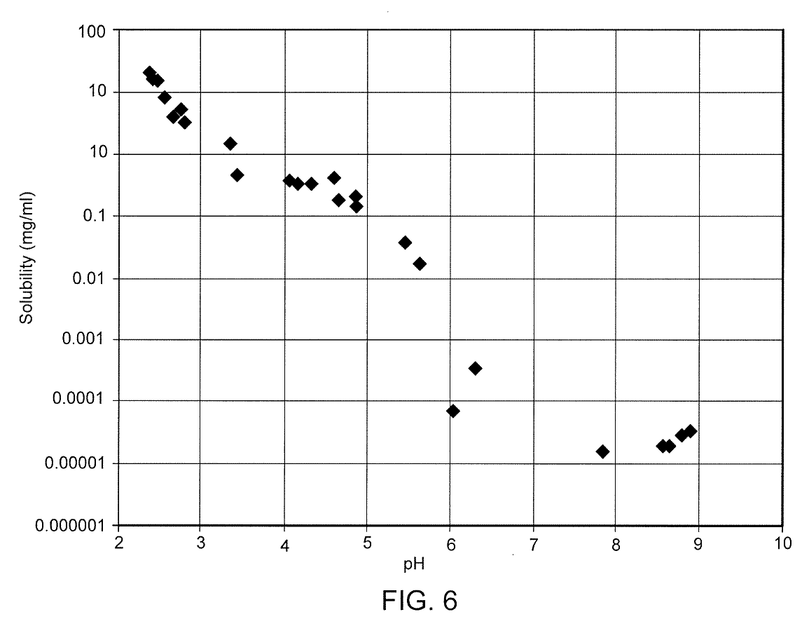

The results of the solubility study are shown in FIG. 6. As shown in FIG. 6, the compound exhibits a pH-dependent solubility, ranging from greater than 1 mg/mL at pH less than about 3 to less than 0.001 mg/mL at pH less than about 6. This pH solubility profile is consistent with calculated pKa values of 7.9, 4.4, and 4.2, based on the Hammett-Taft equation.

### Example 6

### Effect of Citric Acid on Dissolution

The effect of citric acid on the dissolution of one of the compounds described herein was evaluated to determine if the presence of citric acid improved the dissolution performance of the formulated compound. Two sets of formulations were prepared for the citric acid study, with and without citric acid. The formulations also included Surelease sustained release coating.

The first formulation was prepared with coated beads containing approximately 2% 5-fluoro-8-{4-[4-(6-methoxyquinolin-8-yl)piperazin-1-yl]piperidin-1-yl}quinoline by weight and 11% Surelease sustained release coating. Table 5 provides the final composition of the beads.

TABLE 5
Composition of Beads Without Acid

| Ingredient | w/w % |
|---|---|
| Sugar sphere | 85.98 |
| 5-fluoro-8-{4-[4-(6-methoxyquinolin-8- | 3.03^{d} |
| yl)piperazin-1-yl]piperidin-1-yl}quinoline | |
| trisuccinate^{a} | |
| Opadry Clear II | 1.07 |
| Surelease | 9.91 |
| Water^{e} | q.s. |

^{d}Equivalent to 1.72%, based on ~56.6% use at value.
^{e}Not present in final product

The second first formulation was prepared with about 2.5% 5-fluoro-8-{4-[4-(6-methoxyquinolin-8-yl)piperazin-1-yl]piperidin-1-yl}quinoline by weight, about 10.5% by weight Surelease sustained release coating, and about 4% by weight citric acid. Table 6 provides the final composition of the beads.

TABLE 6
Composition of Beads With Acid

| Ingredient | w/w % |
|---|---|
| Sugar sphere | 82.12 |
| 5-fluoro-8-{4-[4-(6-methoxyquinolin-8- | 3.63^{d} |
| yl)piperazin-1-yl]piperidin-1-yl}quinoline | |
| trisuccinate^{a} | |
| Opadry Clear II | 0.64 |
| Citric Acid | 4.11 |
| Surelease | 9.50 |
| Water^{e} | q.s. |

^{d}Equivalent to 2.05%, based on ~56.6% use at value.
^{e}Not present in final product

For each formulation, citric acid and the compound were coated onto sugar spheres with an overcoat of Surelease ethylcellulose sustained-release polymer, in a manner similar to that described above in Example 1. The beads were then loaded into capsules such that each capsule contained about 2.5 mg of the compound.

The dissolution profiles for each formulation were determined using United States Pharmacopoeia (“USP”) Apparatus 2 at 50 and 75 rpm. The formulation containing citric acid was tested using the two-step methodology described above (pH˜1 (0.1N HCl) for approximately two hours, followed by exposure to a buffer of pH˜6.8 containing about 1% SLS for approximately six more hours). The formulations with no citric acid component were tested in two different dissolution media—0.1 N hydrochloric acid and pH 6.8 buffer, with no change in pH during the study. For each dosage form/test condition, two dissolution tests were performed (N=2).

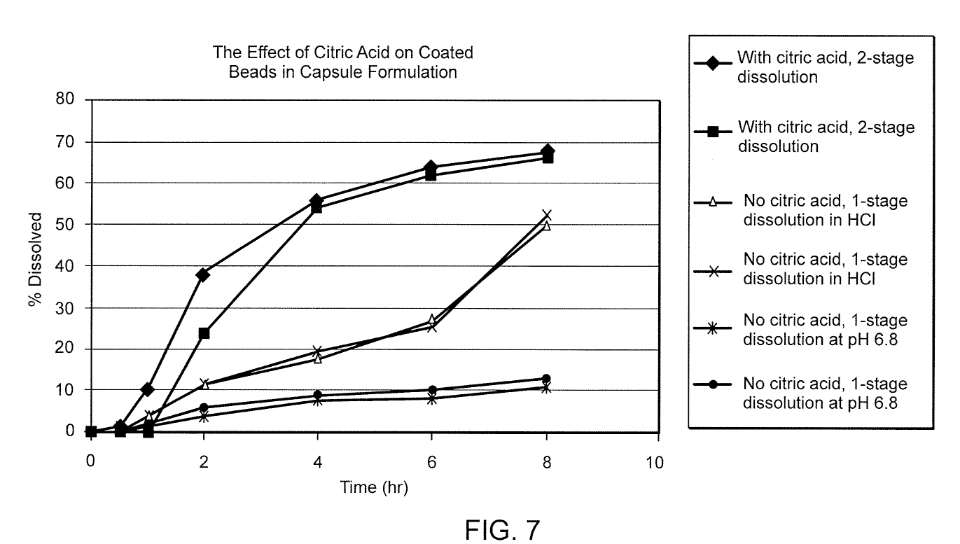

The results of the citric acid study are presented in FIG. 7. As shown in FIG. 7, when compared to the dissolution of the non-acid-containing formulation, in either pH 6.8 or 0.1N HCl, the dissolution of the compound is enhanced in the presence of citric acid.

### Example 7

### Effect of Enteric Coating on Dissolution

The effect of enteric coating on the dissolution of one of the compounds described herein was evaluated. Two groups of formulations were prepared for the citric acid study. The first group included the coated beads of Example 3. In the second group, beads were also coated with approximately 2% of 5-fluoro-8-{4-[4-(6-methoxyquinolin-8-yl)piperazin-1-yl]piperidin-1-yl}quinoline by weight and citric acid, with a coating of 8% Surelease sustained release coating by weight, and an additional coating of 10% enteric coating by weight. The beads were prepared according to procedures similar to those described above in Example 1. The beads were loaded into capsules to a fill weight of about 10 mg. Table 7 provides the final composition of the beads.

TABLE 7
Composition of Beads With Enteric Coating

| Ingredient | w/w % |
|---|---|
| Sugar sphere | 75.08 |
| 5-fluoro-8-{4-[4-(6-methoxyquinolin-8- | 3.32^{d} |
| yl)piperazin-1-yl]piperidin-1-yl}quinoline | |
| trisuccinate^{a} | |
| Opadry Clear II | 0.59 |
| Citric Acid | 3.75 |
| Surelease | 7.41 |
| Eudragit L30D-55 (solid) | 8.27 |
| Triethyl citrate | 0.62 |
| Imwitor 900K | 0.83 |
| Tween 80 | 0.08 |
| NaOH (solid) | 0.04 |
| Water^{e} | q.s. |

^{d}Equivalent to 1.88%, based on ~56.6% use at value.
^{e}Not present in final product

The dissolution profiles for each formulation were determined using the two-stage dissolution methodology described in Example 7. For each formulation, two dissolution tests were performed (N=2).

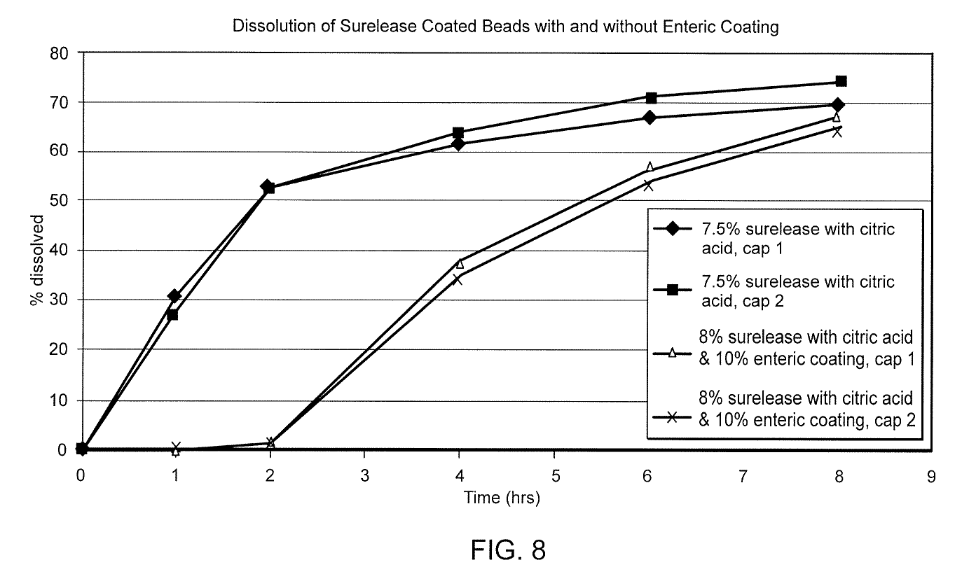

The results are presented in FIG. 8. As shown in FIG. 8, the data demonstrate that by adding an enteric coating to beads already having a sustained release coat, drug dissolution is depressed until a pH corresponding to approximately intestinal pH is reached.

### Example 8

### Effect of Sustained Release Coating on Dissolution

The effect of sustained release coating on the dissolution of one of the compounds described herein was evaluated. Two groups of formulations were prepared for this study. In the first group, beads were coated with approximately 2% of 5-fluoro-8-{4-[4-(6-methoxyquinolin-8-yl)piperazin-1-yl]piperidin-1-yl}quinoline by weight and citric acid and approximately 7.5% Surelease sustained release coating by weight. The beads were loaded into capsules to a fill weight of about 10 mg. Table 8 provides the final composition of the pellets.

TABLE 8
Composition of Beads With Sustained Release Coating (2-layer)

| Ingredient | w/w % |
|---|---|
| Sugar sphere | 84.41 |
| 5-fluoro-8-{4-[4-(6-methoxyquinolin-8- | 3.73^{d} |
| yl)piperazin-1-yl]piperidin-1-yl}quinoline | |
| trisuccinate^{a} | |
| Opadry Clear II | 0.66 |
| Citric Acid | 4.22 |
| Surelease | 6.98 |
| Water^{e} | q.s. |

^{d}Equivalent to 2.11%, based on ~56.6% use at value.
^{e}Not present in final product

The second group contained a mixture of 50% of the sustained release coated beads described above (Table 8) and 50% of the three-layer beads described in Example 2 that contained an additional 10% Eudragit enteric coating. All beads were prepared according to procedures similar to those described above in Example 1.

The dissolution profiles for each formulation were determined using the two-stage dissolution methodology described in Example 7. For each formulation, two dissolution tests were performed (N=2).

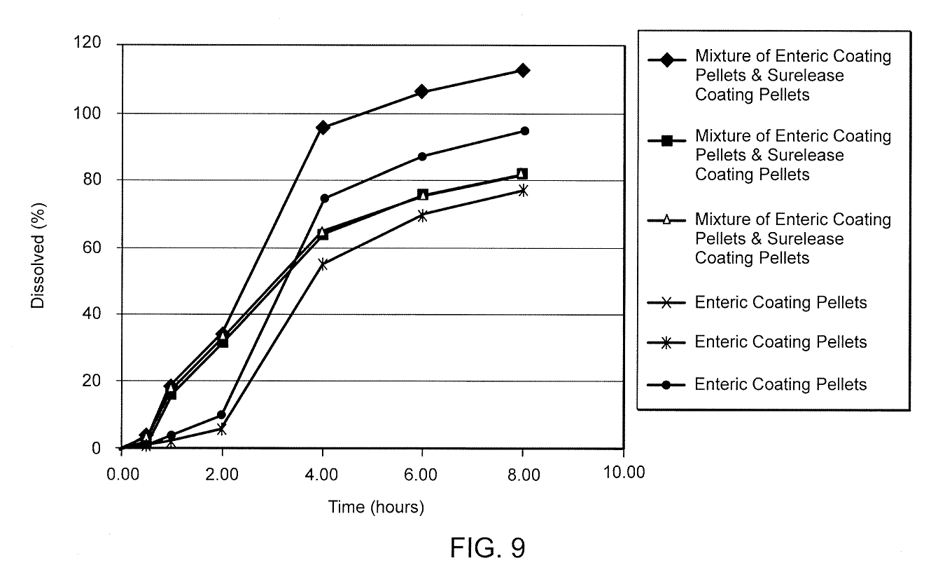

The results are presented in FIG. 9. As shown in FIG. 9, the data demonstrate that the sustained release coating with the enteric coating provides continues drug release throughout the study.

### Example 9

### In Vivo Dog Studies

Pharmaceutical compositions prepared as described above were evaluated in male beagle dogs. In the study, the dogs were divided into two groups of four male beagles each. In Group A, the dogs of Group I were administered capsules prepared according to the procedure as described in Example 1 (i.e., 4-layer capsules). Table 9 provides the final composition of the beads.

TABLE 9
Composition of 4-Layer Coated Beads Used in the Dog Study (Group A)

| Ingredient | w/w % |
|---|---|
| Sugar sphere | 73.070 |
| 5-fluoro-8-{4-[4-(6-methoxyquinolin-8- | 5.380^{d} |
| yl)piperazin-1-yl]piperidin-1-yl}quinoline | |
| trisuccinate^{a} | |
| Opadry Clear II | 1.490 |
| Citric Acid | 1.830 |
| Surelease | 8.830 |
| Eugragit L30D-55 (solid) | 7.890 |
| Triethyl citrate | 0.590 |
| Imwitor 900K | 0.790 |
| Tween 80 | 0.08 |
| NaOH (solid) | 0.05 |
| Water^{e} | q.s. |

^{d}Equivalent to 3.04%, based on ~56.6% use at value.
^{e}Not present in final product

In Group B, the dogs were administered a mixture containing 50% of capsules prepared according to Example 2 and 50% of capsules prepared according to Example 3. Each dog was administered a single dose of approximately 10 mg of the pharmaceutical composition. Following the study, an assay of the pharmaceutical composition confirmed each dose was 10 mg, except for the Group A capsule formulation, which was approximately 8.2 mg. The formulation was administered orally following an overnight fast. Food was made available to the animals four hours following administration and water was available ad libitum over the entire duration of the study. Blood samples were collected pre-dose (0 hour) and at 0.5, 1, 2, 3, 4, 6, 8, 12, 24, 32, and 48 hours after dosing.

The results of the in vivo study confirm the bioavailability of the pharmacological agent, with measurable plasma concentration levels observed through at least twelve hours post-administration. Table 10 provides the area under the curve results for the two formulations studied, a slow and a fast formulation, based on relative dissolution rate. The area under the curve results were normalized for the actual dose delivered in each group. These data demonstrate that the slow capsule formulation provided the numerically highest mean exposure. The slow capsule formulation was also numerically higher than a capsule containing the drug substance that had an AUC0-t/Dose of 446 (ng.hr/mL)/(mg/kg).

TABLE 10
Mean AUC0-t/Dose Values Observed in Dogs Following a Single Oral
Administration of Sustained Release Formulations

| | AUC₀₋ₜ/Dose |
|---|---|
| 10 mg formulation | (ng · hr/mL)/(mg/kg) |
| Group A | 606 |
| Group B | 350 |

Those skilled in the art will readily appreciate that numerous changes and modifications can be made to the embodiments and that such changes and modifications can be made without departing from the spirit of the invention. Those skilled in the art will recognize, or be able to ascertain using no more than routine experimentation, many equivalents to the specific embodiments described herein. It is, therefore, intended that the claims cover all such equivalent variations that fall within the scope of invention.

We claim:

1. A controlled-release bead comprising:
(i) a core unit of a substantially water-soluble or water-swellable inert material;
(ii) a first layer on the core unit comprising a pharmacological agent, an acidifier and optionally a binder;
(iii) a second layer of sustained-release coat covering the first layer;
(iv) a third layer of enteric coat on the second layer; and
(v) optionally, an outermost layer comprising the pharmacological agent and optionally a binder,
wherein the pharmacological agent is a compound of Formula I or a pharmaceutically acceptable salt thereof: 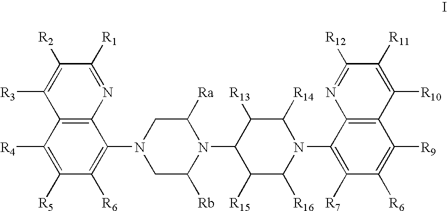
wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₁-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Rₐ and R_{b} are each independently —H or —CH₃; and
R₂₅ is —H, linear or branched (C₁-C₆)-alkyl, (C₁-C₆)-haloalkyl, (C₂-C₆)-alkenyl, or (C₂-C₆)-alkynyl.

2. The controlled-release bead of claim 1, wherein the pharmacological agent is a compound selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
5-fluoro-8-(4-(4-(6-methoxyquinolin-8-yl)piperazin-1-yl)piperidin-1-yl)quinoline trisuccinate;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline;
4,5-difluoro-8-(4-(4-(6-methoxyquinolin-8-yl)piperazin-1-yl)piperidin-1-yl)quinoline;
and pharmaceutically acceptable salts thereof.

3. The controlled-release bead of claim 1, wherein the pharmacological agent is 5-fluoro-8-{4-[4-(6-methoxyquinolin-8-yl)piperazin-1-yl]piperidin-1-yl}quinoline trisuccinate.

4. The controlled-release bead of claim 3, wherein the sustained-release coat is effective for controlled release of the pharmacological agent contained in the first layer or the core unit, wherein the enteric coat is effective for delaying the onset of the release of the pharmacological agent contained in the first layer or the core unit, and wherein the outermost layer is effective for immediate release of the pharmacological agent contained in the outermost layer.

5. The controlled-release bead of claim 3, characterized in that about 15% to about 35% by weight of the pharmacological agent is released after about 2 hours and about 45% to about 65% by weight of the pharmacological agent is released after about 8 hours, in simulated gastrointestinal media.

6. The controlled-release bead of claim 3, characterized in that less than about 15% by weight of the pharmacological agent is released after about 2 hours and more than about 60% by weight of the pharmacological agent is released after about 8 hours, in simulated gastrointestinal media.

7. The controlled-release bead of claim 3, wherein the water-soluble or water-swellable inert material comprises a sphere selected from sucrose, starch, Sugar Spheres NF, sucrose crystals, microcrystalline cellulose, lactose, and mixtures thereof.

8. The controlled-release bead of claim 3, wherein the sustained-release coat comprises polymethacrylate, methacrylic acid-methacrylic acid ester copolymer, acrylate methacrylate copolymer, ethylacrylate/methylmethacrylate copolymer, cellulose acetate, ethylcellulose, high viscosity matrix forming hydroxypropyl methyl cellulose, low viscosity matrix forming hydroxypropyl methyl cellulose, and mixtures thereof.

9. The controlled-release bead of claim 3, wherein the sustained-release coat comprises ethylcellulose.

10. The controlled-release bead of claim 3, wherein the enteric coat comprises an enteric coating polymer or copolymer, an optional pH adjustment agent, an optional glidant, an optional plasticizer, an optional surfactant, and mixtures thereof.

11. The controlled-release bead of claim 10, wherein the enteric coating polymer or copolymer is selected from methacrylic polymer or copolymer, methacrylic acid polymer or copolymer, acrylic copolymer, acrylic acid polymer or copolymer, vinyl polymer or copolymer, hypromellose containing enteric coating system, cellulose acetate phthalate, hydroxypropylmethyl cellulose acetate phthalate, cellulosic polymer, poly(methyl vinyl ether/maleic anhydride), zein, shellac, and mixtures thereof.

12. The controlled-release bead of claim 10, wherein the enteric coating polymer or copolymer is methacrylic copolymer with an anionic functional group.

13. The controlled-release bead of claim 10, wherein the enteric coating polymer or copolymer is selected from methyl methacrylate, ethyl methacrylate and mixtures thereof.

14. The controlled-release bead of claim 10, wherein the enteric coating polymer or copolymer is Eudragit polymer.

15. The controlled-release bead of claim 10, wherein the pH adjustment agent is selected from NaOH, KOH, NH₄OH, and mixtures thereof.

16. The controlled-release bead of claim 10, wherein the glidant is selected from mono- and di-glycerides, talc, silicon dioxide, silicates, stearic acid, starch, cellulose, lactose, stearates, calcium phosphates, magnesium carbonate, magnesium oxide, silicon dioxide aerogels, and mixtures thereof.

17. The controlled-release bead of claim 10, wherein the glidant is selected from mono- and di-glycerides.

18. The controlled-release bead of claim 10, wherein the plasticizer is selected from triethyl citrate, dibutyl sebecate, propylene glycol, triacetin, sorbitol, tributyl citrate, acetyltriethyl citrate, dibutyl phthalate, triethanolamine, diethyl phthalate, acetylated monoglyceride, glycerol, a fatty acid ester, and mixtures thereof.

19. The controlled-release bead of claim 10, wherein the plasticizer is triethyl citrate.

20. The controlled-release bead of claim 10, wherein the surfactant is selected from sodium lauryl sulfate, dioctyl sodium sulfosuccinate, polyoxyethylene alkyl ether, polyoxyethylene alkyl ester, polysorbate, a sugar esters, poloxamer, docusate sodium, polyoxyethylene stearate, sorbitan fatty acid ester, vitamin E TPGS, and mixtures thereof.

21. The controlled-release bead of claim 10, wherein the surfactant is polysorbate.

22. The controlled-release bead of claim 3, wherein the binder is selected from hypromellose, polyvinylpyrrolidone, methylcellulose, hydroxypropyl cellulose, hydroxyethyl cellulose, carboxymethyl cellulose, other celluloses, starches and starch derivatives, polyvinyl alcohol, and mixtures thereof.

23. The controlled-release bead of claim 3, wherein the binder is hypromellose.

24. The controlled-release bead of claim 3, wherein the binder is Opadry II.

25. The controlled-release bead of claim 3, wherein the acidifier improves the in vitro dissolution of the pharmacological agent at a pH level corresponding to the pH of the lower gastrointestinal tract.

26. The controlled-release bead of claim 3, wherein the acidifier is selected from citric acid, ascorbic acid, glutamic acid, tartaric acid, succinic acid, malic acid, erythorbic acid, propionic acid, lactic acid, oleic acid, fumaric acid, benzoic acid, alginic acid, and mixtures thereof.

27. The controlled-release bead of claim 3, wherein the acidifier is citric acid.

28. The controlled-release bead of claim 3, wherein the outermost layer is present and the ratio between the pharmacological agent contained in the outermost layer to that contained in the first layer or the core unit is from about 15% to about 40% w/w.

29. The controlled-release bead of claim 28, wherein the ratio is from about 20% to about 35% w/w.

30. The controlled-release bead of claim 28, wherein the ratio is from about 25% to about 30% w/w.

31. The controlled-release bead of claim 3, wherein:
a) the water-soluble or water-swellable inert material comprises from about 60% to about 90% by weight of the bead;
b) the pharmacological agent comprises from about 1% to about 25% by weight of the bead;
c) the acidifier comprises from about 0.5% to about 10% by weight of the bead;
d) the sustained-release coat comprises from about 1% to about 20% by weight of the bead;
e) the binder comprises from about 0.1% to about 5% by weight of the bead; and
f) the enteric coat comprises from about 0.5% to about 20% by weight of the bead, in which the enteric coat contains from about 0.5% to about 15% of an enteric coating polymer or copolymer by weight of the bead, from about 0.01% to about 2% of a pH adjustment agent by weight of the bead, from about 0.1% to about 5% of a glidant by weight of the bead, from about 0.1% to about 3% of a plasticizer by weight of the bead, and from about 0.01% to about 2% of a surfactant by weight of the bead.

32. The controlled-release bead of claim 31, wherein:
the pharmacological agent comprises from about 1% to about 10% by weight of the bead;
the acidifier comprises from about 1% to about 5% by weight of the bead;
the sustained-release coat comprises from about 5% to about 15% by weight of the bead;
and the enteric coat comprises from about 1% to about 15% by weight of the bead.

33. A multiple particulate formulation comprising a plurality of beads according to claim 3.

34. The multiple particulate formulation of claim 33 which is a capsule or tablet.

35. A controlled-release bead comprising:
(i) a core unit comprising a mixture of a substantially water-soluble or water-swellable inert material, a pharmacological agent, an acidifier and optionally a binder;
(ii) a first layer of sustained-release coat on the core unit;
(iii) a second layer of enteric coat covering the first layer; and
(iv) optionally, an outermost layer comprising the pharmacological agent and optionally a binder,
wherein the pharmacological agent is a compound of Formula I or a pharmaceutically acceptable salt thereof: 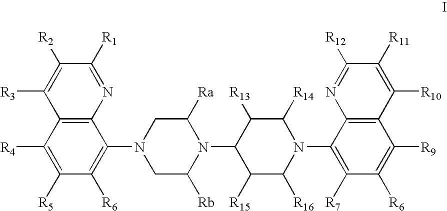
wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Rₐ and R_{b} are each independently —H or —CH₃; and
R₂₅ is —H, linear or branched (C₁-C₆)-alkyl, (C₁-C₆)-haloalkyl, (C₂-C₆)-alkenyl, or (C₂-C₆)-alkynyl.

36. The controlled-release bead of claim 35, wherein the pharmacological agent is a compound selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
5-fluoro-8-(4-(4-(6-methoxyquinolin-8-yl)piperazin-1-yl)piperidin-1-yl)quinoline trisuccinate;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline;
4,5-difluoro-8-(4-(4-(6-methoxyquinolin-8-yl)piperazin-1-yl)piperidin-1-yl)quinoline;
and pharmaceutically acceptable salts thereof.

37. The controlled-release bead of claim 35, wherein the pharmacological agent is 5-fluoro-8-{4-[4-(6-methoxyquinolin-8-yl)piperazin-1-yl]piperidin-1-yl}quinoline trisuccinate.

38. The controlled-release bead of claim 37, wherein the sustained-release coat is effective for controlled release of the pharmacological agent contained in the first layer or the core unit, wherein the enteric coat is effective for delaying the onset of the release of the pharmacological agent contained in the first layer or the core unit, and wherein the outermost layer is effective for immediate release of the pharmacological agent contained in the outermost layer.

39. The controlled-release bead of claim 37, characterized in that about 15% to about 35% by weight of the pharmacological agent is released after about 2 hours and about 45% to about 65% by weight of the pharmacological agent is released after about 8 hours, in simulated gastrointestinal media.

40. The controlled-release bead of claim 37, characterized in that less than about 15% by weight of the pharmacological agent is released after about 2 hours and more than about 60% by weight of the pharmacological agent is released after about 8 hours, in simulated gastrointestinal media.

41. The controlled-release bead of claim 37, wherein the water-soluble or water-swellable inert material comprises a sphere selected from sucrose, starch, Sugar Spheres NF, sucrose crystals, microcrystalline cellulose, lactose, and mixtures thereof.

42. The controlled-release bead of claim 37, wherein the sustained-release coat comprises polymethacrylate, methacrylic acid-methacrylic acid ester copolymer, acrylate methacrylate copolymer, ethylacrylate/methylmethacrylate copolymer, cellulose acetate, ethylcellulose, high viscosity matrix forming hydroxypropyl methyl cellulose, low viscosity matrix forming hydroxypropyl methyl cellulose, and mixtures thereof.

43. The controlled-release bead of claim 37, wherein the sustained-release coat comprises ethylcellulose.

44. The controlled-release bead of claim 37, wherein the enteric coat comprises an enteric coating polymer or copolymer, an optional pH adjustment agent, an optional glidant, an optional plasticizer, an optional surfactant, and mixtures thereof.

45. The controlled-release bead of claim 44, wherein the enteric coating polymer or copolymer is selected from methacrylic polymer or copolymer, methacrylic acid polymer or copolymer, acrylic copolymer, acrylic acid polymer or copolymer, vinyl polymer or copolymer, hypromellose containing enteric coating system, cellulose acetate phthalate, hydroxypropylmethyl cellulose acetate phthalate, cellulosic polymer, poly(methyl vinyl ether/maleic anhydride), zein, shellac, and mixtures thereof.

46. The controlled-release bead of claim 44, wherein the enteric coating polymer or copolymer is methacrylic copolymer with an anionic functional group.

47. The controlled-release bead of claim 44, wherein the enteric coating polymer or copolymer is selected from methyl methacrylate, ethyl methacrylate and mixtures thereof.

48. The controlled-release bead of claim 44, wherein the enteric coating polymer or copolymer is Eudragit polymer.

49. The controlled-release bead of claim 44, wherein the pH adjustment agent is selected from NaOH, KOH, NH₄OH, and mixtures thereof.

50. The controlled-release bead of claim 44, wherein the glidant is selected from mono- and di-glycerides, talc, silicon dioxide, silicates, stearic acid, starch, cellulose, lactose, stearates, calcium phosphates, magnesium carbonate, magnesium oxide, silicon dioxide aerogels, and mixtures thereof.

51. The controlled-release bead of claim 44, wherein the glidant is selected from mono- and di-glycerides.

52. The controlled-release bead of claim 44, wherein the plasticizer is selected from triethyl citrate, dibutyl sebecate, propylene glycol, triacetin, sorbitol, tributyl citrate, acetyltriethyl citrate, dibutyl phthalate, triethanolamine, diethyl phthalate, acetylated monoglyceride, glycerol, a fatty acid ester, and mixtures thereof.

53. The controlled-release bead of claim 44, wherein the plasticizer is triethyl citrate.

54. The controlled-release bead of claim 44, wherein the surfactant is selected from sodium lauryl sulfate, dioctyl sodium sulfosuccinate, polyoxyethylene alkyl ether, polyoxyethylene alkyl ester, polysorbate, a sugar esters, poloxamer, docusate sodium, polyoxyethylene stearate, sorbitan fatty acid ester, vitamin E TPGS, and mixtures thereof.

55. The controlled-release bead of claim 44, wherein the surfactant is polysorbate.

56. The controlled-release bead of claim 37, wherein the binder is selected from hypromellose, polyvinylpyrrolidone, methylcellulose, hydroxypropyl cellulose, hydroxyethyl cellulose, carboxymethyl cellulose, other celluloses, starches and starch derivatives, polyvinyl alcohol, and mixtures thereof.

57. The controlled-release bead of claim 37, wherein the binder is hypromellose.

58. The controlled-release bead of claim 37, wherein the binder is Opadry II.

59. The controlled-release bead of claim 37, wherein the acidifier improves the in vitro dissolution of the pharmacological agent at a pH level corresponding to the pH of the lower gastrointestinal tract.

60. The controlled-release bead of claim 37, wherein the acidifier is selected from citric acid, ascorbic acid, glutamic acid, tartaric acid, succinic acid, malic acid, erythorbic acid, propionic acid, lactic acid, oleic acid, fumaric acid, benzoic acid, alginic acid, and mixtures thereof.

61. The controlled-release bead of claim 37, wherein the acidifier is citric acid.

62. The controlled-release bead of claim 37, wherein the outermost layer is present and the ratio between the pharmacological agent contained in the outermost layer to that contained in the first layer or the core unit is from about 15% to about 40% w/w.

63. The controlled-release bead of claim 62, wherein the ratio is from about 20% to about 35% w/w.

64. The controlled-release bead of claim 62, wherein the ratio is from about 25% to about 30% w/w.

65. The controlled-release bead of claim 37, wherein:
a) the water-soluble or water-swellable inert material comprises from about 60% to about 90% by weight of the bead;
b) the pharmacological agent comprises from about 1% to about 25% by weight of the bead;
c) the acidifier comprises from about 0.5% to about 10% by weight of the bead;
d) the sustained-release coat comprises from about 1% to about 20% by weight of the bead;
e) the binder comprises from about 0.1% to about 5% by weight of the bead; and
f) the enteric coat comprises from about 0.5% to about 20% by weight of the bead, in which the enteric coat contains from about 0.5% to about 15% of an enteric coating polymer or copolymer by weight of the bead, from about 0.01% to about 2% of a pH adjustment agent by weight of the bead, from about 0.1% to about 5% of a glidant by weight of the bead, from about 0.1% to about 3% of a plasticizer by weight of the bead, and from about 0.01% to about 2% of a surfactant by weight of the bead.

66. The controlled-release bead of claim 65, wherein:
the pharmacological agent comprises from about 1% to about 10% by weight of the bead;
the acidifier comprises from about 1% to about 5% by weight of the bead;
the sustained-release coat comprises from about 5% to about 15% by weight of the bead;
and the enteric coat comprises from about 1% to about 15% by weight of the bead.

67. A multiple particulate formulation comprising a plurality of beads according to claim 37.

68. The multiple particulate formulation of claim 67 which is a capsule or tablet.

69. A multiple particulate formulation comprising:
(A) at least one first bead comprising:
(i) a core unit of a substantially water-soluble or water-swellable inert material;
(ii) a first layer on the core unit comprising a pharmacological agent, an acidifier, and an optional binder;
(iii) a second layer of sustained-release coat covering the first layer; and
(iv) a third layer of enteric coat on the second layer; and
(B) at least one second bead comprising the pharmacological agent optionally covered by a sustained-release coat,
wherein the pharmacological agent is a compound of Formula I or a pharmaceutically acceptable salt thereof: 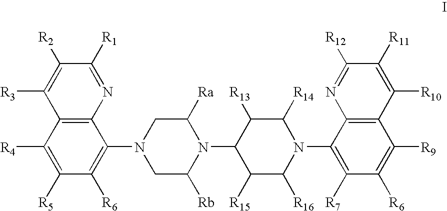
wherein R₁, R₂, R₃, R₄, R₅, R₆, R₇, R₈, R₉, R₁₀, R₁₁, R₁₂, R₁₃, R₁₄, R₁₅, and R₁₆, are each independently —H. (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Rₐ and R_{b} are each independently —H or —CH₃; and
R₂₅ is —H, linear or branched (C₁-C₆)-alkyl, (C₁-C₆)-haloalkyl, (C₂-C₆)-alkenyl, or (C₂-C₆)-alkynyl.

70. The multiple particulate formulation of claim 69, wherein the pharmacological agent is a compound selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
5-fluoro-8-(4-(4-(6-methoxyquinolin-8-yl)piperazin-1-yl)piperidin-1-yl)quinoline trisuccinate;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline;
4,5-difluoro-8-(4-(4-(6-methoxyquinolin-8-yl)piperazin-1-yl)piperidin-1-yl)quinoline;
and pharmaceutically acceptable salts thereof.

71. The multiple particulate formulation of claim 69, wherein the pharmacological agent is 5-fluoro-8-{4-[4-(6-methoxyquinolin-8-yl)piperazin-1-yl]piperidin-1-yl}quinoline trisuccinate.

72. The multiple particulate formulation of claim 71, wherein the sustained-release coat is effective for controlled release of the pharmacological agent, wherein the enteric coat is effective for delaying the onset of the release of the pharmacological agent contained in the first bead, and wherein second bead is effective for immediate release of the pharmacological agent contained in the second bead.

73. The multiple particulate formulation of claim 71, characterized in that about 20% to about 45% by weight of the pharmacological agent is released after about 2 hours and more than about 60% by weight of the pharmacological agent is released after about 8 hours, in simulated gastrointestinal media.

74. The multiple particulate formulation of claim 71, wherein the water-soluble or water-swellable inert material comprises a sphere selected from sucrose, starch, Sugar Spheres NF, sucrose crystals, microcrystalline cellulose, lactose, and mixtures thereof.

75. The multiple particulate formulation of claim 71, wherein the sustained-release coat comprises polymethacrylate, methacrylic acid-methacrylic acid ester copolymer, acrylate methacrylate copolymer, ethylacrylate/methylmethacrylate copolymer, cellulose acetate, ethylcellulose, high viscosity matrix forming hydroxypropyl methyl cellulose, low viscosity matrix forming hydroxypropyl methyl cellulose, and mixtures thereof.

76. The multiple particulate formulation of claim 71, wherein the sustained-release coat comprises ethylcellulose.

77. The multiple particulate formulation of claim 71, wherein the enteric coat comprises an enteric coating polymer or copolymer, an optional pH adjustment agent, an optional glidant, an optional plasticizer, an optional surfactant, and mixtures thereof.

78. The multiple particulate formulation of claim 77, wherein the enteric coating polymer or copolymer is selected from methacrylic polymer or copolymer, methacrylic acid polymer or copolymer, acrylic copolymer, acrylic acid polymer or copolymer, vinyl polymer or copolymer, hypromellose containing enteric coating system, cellulose acetate phthalate, hydroxypropylmethyl cellulose acetate phthalate, cellulosic polymer, poly(methyl vinyl ether/maleic anhydride), zein, shellac, and mixtures thereof.

79. The multiple particulate formulation of claim 77, wherein the enteric coating polymer or copolymer is methacrylic copolymer with an anionic functional group.

80. The multiple particulate formulation of claim 77, wherein the enteric coating polymer or copolymer is selected from methyl methacrylate, ethyl methacrylate and mixtures thereof

81. The multiple particulate formulation of claim 77, wherein the enteric coating polymer or copolymer is Eudragit polymer.

82. The multiple particulate formulation of claim 77, wherein the pH adjustment agent is selected from NaOH, KOH, NH₄OH, and mixtures thereof.

83. The multiple particulate formulation of claim 77, wherein the glidant is selected from mono- and di-glycerides, talc, silicon dioxide, silicates, stearic acid, starch, cellulose, lactose, stearates, calcium phosphates, magnesium carbonate, magnesium oxide, silicon dioxide aerogels, and mixtures thereof.

84. The multiple particulate formulation of claim 77, wherein the glidant is selected from mono- and di-glycerides.

85. The multiple particulate formulation of claim 77, wherein the plasticizer is selected from triethyl citrate, dibutyl sebecate, propylene glycol, triacetin, sorbitol, tributyl citrate, acetyltriethyl citrate, dibutyl phthalate, triethanolamine, diethyl phthalate, acetylated monoglyceride, glycerol, a fatty acid ester, and mixtures thereof.

86. The multiple particulate formulation of claim 77, wherein the plasticizer is triethyl citrate.

87. The multiple particulate formulation of claim 77, wherein the surfactant is selected from sodium lauryl sulfate, dioctyl sodium sulfosuccinate, polyoxyethylene alkyl ether, polyoxyethylene alkyl ester, polysorbate, a sugar esters, poloxamer, docusate sodium, polyoxyethylene stearate, sorbitan fatty acid ester, vitamin E TPGS, and mixtures thereof.

88. The multiple particulate formulation of claim 77, wherein the surfactant is polysorbate.

89. The multiple particulate formulation of claim 77, wherein the binder is selected from hypromellose, polyvinylpyrrolidone, methylcellulose, hydroxypropyl cellulose, hydroxyethyl cellulose, carboxymethyl cellulose, other celluloses, starches and starch derivatives, polyvinyl alcohol, and mixtures thereof.

90. The multiple particulate formulation of claim 77, wherein the binder is hypromellose.

91. The multiple particulate formulation of claim 77, wherein the binder is Opadry II.

92. The multiple particulate formulation of claim 77, wherein the acidifier improves the in vitro dissolution of the pharmacological agent at a pH level corresponding to the pH of the lower gastrointestinal tract.

93. The multiple particulate formulation of claim 77, wherein the acidifier is selected from citric acid, ascorbic acid, glutamic acid, tartaric acid, succinic acid, malic acid, erythorbic acid, propionic acid, lactic acid, oleic acid, fumaric acid, benzoic acid, alginic acid, and mixtures thereof.

94. The multiple particulate formulation of claim 77, wherein the acidifier is citric acid.

95. The multiple particulate formulation of claim 77, wherein the ratio between the pharmacological agent contained in the second bead to that contained in the first bead is from about 15% to about 40% w/w.

96. The multiple particulate formulation of claim 95, wherein the ratio is from about 20% to about 35% w/w.

97. The multiple particulate formulation of claim 95, wherein the ratio is from about 25% to about 30% w/w.

98. The multiple particulate formulation of claim 71, wherein:
a) the water-soluble or water-swellable inert material comprises from about 60% to about 90% by weight of the total formulation;
b) the pharmacological agent comprises from about 1% to about 25% by weight of the total formulation;
c) the acidifier comprises from about 0.5% to about 10% by weight of the total formulation d;
d) the sustained-release coat comprises from about 1% to about 20% by weight of the total formulation;
e) the binder comprises from about 0.1% to about 5% by weight of the total formulation; and
f) the enteric coat comprises from about 0.5% to about 20% by weight of the total formulation, in which the enteric coat contains from about 0.5% to about 15% of an enteric coating polymer or copolymer by weight of the bead, from about 0.01% to about 2% of a pH adjustment agent by weight of the bead, from about 0.1% to about 5% of a glidant by weight of the bead, from about 0.1% to about 3% of a plasticizer by weight of the bead, and from about 0.01% to about 2% of a surfactant by weight of the bead.

99. The multiple particulate formulation of claim 98, wherein:
the pharmacological agent comprises from about 1% to about 10% by weight of the total formulation;
the acidifier comprises from about 1% to about 5% by weight of the total formulation;
the sustained-release coat comprises from about 5% to about 15% by weight of the total formulation;
and the enteric coat comprises from about 1% to about 15% by weight of the total formulation.

100. The multiple particulate formulation of claim 71, wherein the formulation contains from about 0.1 mg to about 100 mg of the pharmacological agent.

101. The multiple particulate formulation of claim 71, wherein the formulation contains from about 0.5 mg to about 25 mg of the pharmacological agent.

102. The multiple particulate formulation of claim 71 which is a capsule or tablet.

103. A multiple particulate formulation comprising:
(A) at least one first bead comprising:
(i) a core unit comprising a mixture of a substantially water-soluble or water-swellable inert material, a pharmacological agent, an acidifier and an optional binder;
(ii) a first layer of sustained-release coat on the core unit; and
(iii) a second layer of enteric coat covering the first layer; and
(B) at least one second bead comprising the pharmacological agent optionally covered by a sustained-release coat,
wherein the pharmacological agent is a compound of Formula I or a pharmaceutically acceptable salt thereof: 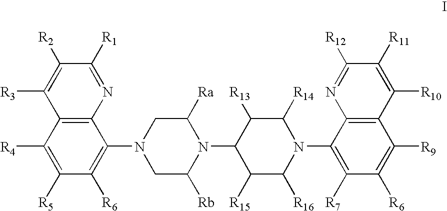
wherein R₁, R₂, R_{3l , R}₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Rₐ and R_{b} are each independently —H or —CH₃; and
R₂₅ is —H, linear or branched (C1-C₆)-alkyl, (C₁-C₆)-haloalkyl, (C₂-C₆)-alkenyl, or (C₂-C₆)-alkynyl.

104. The multiple particulate formulation of claim 103, wherein the pharmacological agent is a compound selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
5-fluoro-8-(4-(4-(6-methoxyquinolin-8-yl)piperazin-1-yl)piperidin-1-yl)quinoline trisuccinate;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline;
4,5-difluoro-8-(4-(4-(6-methoxyquinolin-8-yl)piperazin-1-yl)piperidin-1-yl)quinoline;
and pharmaceutically acceptable salts thereof.

105. The multiple particulate formulation of claim 103, wherein the pharmacological agent is 5-fluoro-8-{4-[4-(6-methoxyquinolin-8-yl)piperazin-1-yl]piperidin-1-yl}quinoline trisuccinate.

106. The multiple particulate formulation of claim 105, wherein the sustained-release coat is effective for controlled release of the pharmacological agent, wherein the enteric coat is effective for delaying the onset of the release of the pharmacological agent contained in the first bead, and wherein second bead is effective for immediate release of the pharmacological agent contained in the second bead.

107. The multiple particulate formulation of claim 105, characterized in that about 20% to about 45% by weight of the pharmacological agent is released after about 2 hours and more than about 60% by weight of the pharmacological agent is released after about 8 hours, in simulated gastrointestinal media.

108. The multiple particulate formulation of claim 105, wherein the water-soluble or water-swellable inert material comprises a sphere selected from sucrose, starch, Sugar Spheres NF, sucrose crystals, microcrystalline cellulose, lactose, and mixtures thereof.

109. The multiple particulate formulation of claim 105, wherein the sustained-release coat comprises polymethacrylate, methacrylic acid-methacrylic acid ester copolymer, acrylate methacrylate copolymer, ethylacrylate/methylmethacrylate copolymer, cellulose acetate, ethylcellulose, high viscosity matrix forming hydroxypropyl methyl cellulose, low viscosity matrix forming hydroxypropyl methyl cellulose, and mixtures thereof.

110. The multiple particulate formulation of claim 105, wherein the sustained-release coat comprises ethylcellulose.

111. The multiple particulate formulation of claim 105, wherein the enteric coat comprises an enteric coating polymer or copolymer, an optional pH adjustment agent, an optional glidant, an optional plasticizer, an optional surfactant, and mixtures thereof.

112. The multiple particulate formulation of claim 111, wherein the enteric coating polymer or copolymer is selected from methacrylic polymer or copolymer, methacrylic acid polymer or copolymer, acrylic copolymer, acrylic acid polymer or copolymer, vinyl polymer or copolymer, hypromellose containing enteric coating system, cellulose acetate phthalate, hydroxypropylmethyl cellulose acetate phthalate, cellulosic polymer, poly(methyl vinyl ether/maleic anhydride), zein, shellac, and mixtures thereof.

113. The multiple particulate formulation of claim 111, wherein the enteric coating polymer or copolymer is methacrylic copolymer with an anionic functional group.

114. The multiple particulate formulation of claim 111, wherein the enteric coating polymer or copolymer is selected from methyl methacrylate, ethyl methacrylate and mixtures thereof.

115. The multiple particulate formulation of claim 111, wherein the enteric coating polymer or copolymer is Eudragit polymer.

116. The multiple particulate formulation of claim 111, wherein the pH adjustment agent is selected from NaOH, KOH, NH₄OH, and mixtures thereof.

117. The multiple particulate formulation of claim 111, wherein the glidant is selected from mono- and di-glycerides, talc, silicon dioxide, silicates, stearic acid, starch, cellulose, lactose, stearates, calcium phosphates, magnesium carbonate, magnesium oxide, silicon dioxide aerogels, and mixtures thereof.

118. The multiple particulate formulation of claim 111, wherein the glidant is selected from mono- and di-glycerides.

119. The multiple particulate formulation of claim 111, wherein the plasticizer is selected from triethyl citrate, dibutyl sebecate, propylene glycol, triacetin, sorbitol, tributyl citrate, acetyltriethyl citrate, dibutyl phthalate, triethanolamine, diethyl phthalate, acetylated monoglyceride, glycerol, a fatty acid ester, and mixtures thereof.

120. The multiple particulate formulation of claim 111, wherein the plasticizer is triethyl citrate.

121. The multiple particulate formulation of claim 111, wherein the surfactant is selected from sodium lauryl sulfate, dioctyl sodium sulfosuccinate, polyoxyethylene alkyl ether, polyoxyethylene alkyl ester, polysorbate, a sugar esters, poloxamer, docusate sodium, polyoxyethylene stearate, sorbitan fatty acid ester, vitamin E TPGS, and mixtures thereof.

122. The multiple particulate formulation of claim 111, wherein the surfactant is polysorbate.

123. The multiple particulate formulation of claim 105, wherein the binder is selected from hypromellose, polyvinylpyrrolidone, methylcellulose, hydroxypropyl cellulose, hydroxyethyl cellulose, carboxymethyl cellulose, other celluloses, starches and starch derivatives, polyvinyl alcohol, and mixtures thereof.

124. The multiple particulate formulation of claim 105, wherein the binder is hypromellose.

125. The multiple particulate formulation of claim 105, wherein the binder is Opadry II.

126. The multiple particulate formulation of claim 105, wherein the acidifier improves the in vitro dissolution of the pharmacological agent at a pH level corresponding to the pH of the lower gastrointestinal tract.

127. The multiple particulate formulation of claim 105, wherein the acidifier is selected from citric acid, ascorbic acid, glutamic acid, tartaric acid, succinic acid, malic acid, erythorbic acid, propionic acid, lactic acid, oleic acid, fumaric acid, benzoic acid, alginic acid, and mixtures thereof.

128. The multiple particulate formulation of claim 105, wherein the acidifier is citric acid.

129. The multiple particulate formulation of claim 105, wherein the ratio between the pharmacological agent contained in the second bead to that contained in the first bead is from about 15% to about 40% w/w.

130. The multiple particulate formulation of claim 129, wherein the ratio is from about 20% to about 35% w/w.

131. The multiple particulate formulation of claim 129, wherein the ratio is from about 25% to about 30% w/w.

132. The multiple particulate formulation of claim 105, wherein:
a) the water-soluble or water-swellable inert material comprises from about 60% to about 90% by weight of the total formulation;
b) the pharmacological agent comprises from about 1% to about 25% by weight of the total formulation;
c) the acidifier comprises from about 0.5% to about 10% by weight of the total formulation d;
d) the sustained-release coat comprises from about 1% to about 20% by weight of the total formulation;
e) the binder comprises from about 0.1% to about 5% by weight of the total formulation; and
f) the enteric coat comprises from about 0.5% to about 20% by weight of the total formulation, in which the enteric coat contains from about 0.5% to about 15% of an enteric coating polymer or copolymer by weight of the bead, from about 0.01% to about 2% of a pH adjustment agent by weight of the bead, from about 0.1% to about 5% of a glidant by weight of the bead, from about 0.1% to about 3% of a plasticizer by weight of the bead, and from about 0.01% to about 2% of a surfactant by weight of the bead.

133. The multiple particulate formulation of claim 132, wherein:
the pharmacological agent comprises from about 1% to about 10% by weight of the total formulation;
the acidifier comprises from about 1% to about 5% by weight of the total formulation;
the sustained-release coat comprises from about 5% to about 15% by weight of the total formulation;
and the enteric coat comprises from about 1% to about 15% by weight of the total formulation.

134. The multiple particulate formulation of claim 105, wherein the formulation contains from about 0.1 mg to about 100 mg of the pharmacological agent.

135. The multiple particulate formulation of claim 105, wherein the formulation contains from about 0.5 mg to about 25 mg of the pharmacological agent.

136. The multiple particulate formulation of claim 105 which is a capsule or tablet.

137. A method for preparing a controlled-release bead according to claim 1, the method comprising:
(a) providing a core unit of a substantially water-soluble or water-swellable inert material;
(b) applying a first layer comprising a pharmacological agent, an acidifier and optionally a binder to the core unit;
(c) applying a second layer of sustained-release coat to cover the first layer;
(d) applying a third layer of enteric coat onto the second layer; and
(e) optionally, applying an outermost layer comprising the pharmacological agent and optionally a binder onto the third layer.

138. A method for preparing a controlled-release bead according to claim 35, the method comprising:
(a) providing a core unit by combining a substantially water-soluble or water-swellable inert material with a pharmacological agent, an acidifier and optionally a binder;
(c) applying a first layer of sustained-release coat to cover the core unit;
(d) applying a second layer of enteric coat onto the first layer; and
(e) optionally, applying an outermost layer comprising the pharmacological agent and optionally a binder onto the second layer.

139. The method of claim 137, further comprising filling a capsule with a plurality of beads of claim 1 to achieve a predetermined dose of the pharmacological agent.

140. The method of claim 138, further comprising filling a capsule with a plurality of beads of claim 35 to achieve a predetermined dose of the pharmacological agent.

141. A method for treating a 5-HT_{1A}-related disorder to a patient in need thereof, the method comprising administering to the patient a therapeutically effective amount of a multiple particulate formulation as defined claim 33.

142. The method of claim 141, wherein the 5-HT_{1A}-related disorder is a cognition-related disorder or an anxiety-related disorder.

143. The method of claim 142, wherein the cognition-related disorder is dementia, Parkinson's disease, Huntington's disease, Alzheimer's disease, cognitive deficits associated with Alzheimer's disease, mild cognitive impairment, or schizophrenia.

144. The method of claim 142, wherein the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.
